(19) United States
(12) Patent Application Publication
(10) Pub. No.: US 2024/0102976 A1
(43) Pub. Date: Mar. 28, 2024

(54) METHOD FOR SCREENING PESTICIDE RESIDUES IN MEDICINE FOOD HOMOLOGY CHARACTERISTIC AGRICULTUREAL PRODUCTS

(71) Applicant: Changchun Customs Technology Center, Changchun (CN)

(72) Inventors: Xun Zhang, Changchun (CN); Tao Liu, Changchun (CN); TingTing Hu, Changchun (CN); JiaHui Wang, Changchun (CN); Yao Fu, Changchun (CN); GuangYu Chen, Changchun (CN); XueSong Chen, Changchun (CN); Xue Jiang, Changchun (CN)

(21) Appl. No.: 18/469,539

(22) Filed: Sep. 18, 2023

(30) Foreign Application Priority Data
Sep. 23, 2022 (CN) 2022111654564

(51) Int. Cl. G01N 30/72; G01N 30/60; G16C 20/62; G16C 20/64

(52) U.S. Cl. CPC G01N 30/7266; G01N 30/60; G16C 20/62; G16C 20/64; G01N 2030/027

(57) ABSTRACT

A method for screening pesticide residues in medicine food homology (MFH) characteristic agricultural products includes step 1, a liquid chromatography-mass spectrometry database of pesticides is constructed, standard solution are prepared for the pesticides and liquid chromatography and mass spectrometry are used to detect the standard solutions; step 2, a sample is processed and then is detected to obtain a liquid chromatography-mass spectrogram; step 3, the liquid chromatography-mass spectrogram obtained from step 2 and liquid chromatography-mass spectrograms in the liquid chromatography-mass spectrometry database obtained from step 1 are compared and analyzed individually. The advances are as follows: a high-resolution rapid method for no less than 300 pesticide residues has been established, the method has high qualitative accuracy, controls the probability of quality accidents to almost zero, and provides reliable technical support for customs supervision of import and export of the MFH agricultural products.

### TECHNICAL FIELD

The disclosure relates to the technical field of pharmaceutical analysis, and particularly to a method for screening pesticide residues in medicine food homology (MFH) characteristic agricultural products.

### BACKGROUND

MFH products are a health food produced based on the national catalog of medicinal food homology, which is between health products and medicines. The MFH products have both the therapeutic effect of medicines and the safety and stability of food. The MFH agricultural products include ginseng, acanthopanax senticosus, epimedium, and Ganoderma lucidum.

In recent years, safety issues related to the MFH products have been frequently exposed, among which poisoning incidents caused by excessive pesticide residues are particularly serious. There are many kinds of pesticides with different chemical structures and properties, as well as complex sample matrices, so methods for detecting the pesticides require precise operational means, high sensitivity, and strong specificity. At present, a multi-residue analysis technology is often used to detect agricultural and veterinary medicine residues in MFH agricultural products, but the detection items are numerous and the sample matrixes are complex. The main detection techniques used based on national, industry, and local standards are gas chromatography-mass spectrum/mass spectrometry and liquid chromatography-mass spectrum/mass spectrometry, and to ensure the specificity of the detection methods, a tandem mass spectrometry technology is often used. With the annual growth of Chinese bureau's detection business and the urgent need for safety detection of the MFH agricultural products at home and abroad, it is urgent to establish a fast and accurate screening and detection method for agricultural and veterinary medicine residues in food. The U.S. Food and Drug Administration (FDA) began research and development on high-resolution screening of pesticides in 2010. Due to the high selectivity of high-resolution mass spectrometry, the anti-interference ability is further enhanced, and it can be accurately and reliably applied to rapid screening and confirmation of target compounds related to food safety, as well as high sensitivity and high flux multi-residue analysis. With the annual growth of detection business of the MFH agricultural products exported from China, and the urgent need for safety detection of the MFH agricultural products at home and abroad, it is urgent to establish a rapid and accurate screening and detection method for pesticide residues in the MFH agricultural products.

### SUMMARY

In order to establish a rapid and accurate detection method for pesticide residues in medicine food homology (MFH) agricultural products, the disclosure provides a method for screening pesticide residues in the MFH characteristic agricultural products.

The method for screening the pesticide residues in the MFH characteristic agricultural products includes the following steps:
step 1, a liquid chromatography-mass spectrometry database of pesticides is constructed. Standard solutions are prepared for the pesticides and liquid chromatography and mass spectrometry are used to detect the standard solutions, a detection condition of the liquid chromatography includes:
   a liquid chromatography column: an Accucore™ aQ C18 hydrophilic interaction liquid chromatography (HPLC) column, 150 millimeter (mm)×2.1 mm (internal diameter, i.d.), a particle size of 2.6 micrometer (μm);
   a mobile phase A: ammonium formate of 5 millimol per liter (mM) and a 0.1% formic acid aqueous solution;
   a mobile phase B: ammonium formate of 5 Mm and a 0.1% formic acid methanol solution
   a column temperature: 25° C., and
a detection condition of the mass spectrometry includes:
   an ion source: an electrospray ionization;
   a temperature of the ion source: 325° C.;
   a temperature of a heater: 350° C.;
   a sheath gas flow rate: 40 atmospheres relative to vacuum (arb);
   an aux gas flow rate: 10 arb,
step 2, a sample is processed to obtain a processed sample, and the processed sample is detected to obtain a liquid chromatography-mass spectrogram of the processed sample;
step 3, the liquid chromatography-mass spectrogram obtained from step 2 and the liquid chromatography-mass spectrograms in the liquid chromatography-mass spectrometry database obtained from step 1 are compared and analyzed individually.

The liquid chromatography-mass spectrometry database includes: names of compounds, molecular formulas of the compounds, accurate masses of the compounds, retention times of the compounds and accurate masses of characteristic ion fragments.

The pesticides in step 1 includes acephate, acetamiprid, aminocarb, azamethiphos, benalaxyl, bendiocarb, benfuracarb, bensulide, benzoximate, bifenazate, bupirimate, butafenacil, butoxycarboxim, buturon, carpropamid, carbaryl, carbendazim, carbetamide, carbofuran, carboxin, clethodim, clothianidin, cyazofamid, cycloxydim, cyprodinil, desmedipham, dicrotophos, diethofencarb, dimethoate, dinotefuran, dioxycarb, ethiofencarb, ethirimol, ethofumesate, fenazaquin, fenhexamid, fenobucarb, fenoxycarb, fenpropidine, flufenacet, fuberidazole, furalaxyl, furathiocarb, hexythiazox, hydramethylnon, imidachloprid, indoxacarb, isoprocarb, mefenacet, mepanipyrim, mepronil, metalaxyl, methamidophos, methomyl, methoxyfenozide, m-tolyl methylcarbamate, metribuzin, napropamide, nitenpyram, nuarimol, omethoate, oxamyl, pencycuron, primicarb, propamocarb, propargite, propoxur, prosulfocarb, pyrimethanil, quinoxyfen, rotenone, spirodiclofen, tebufenozide, tebutam, temephos, thiabendazole, thiacloprid, thiamethoxam, thiobencarb, thiodicarb, thiophanate-methyl, trichlorfon, vamidothion, ametryn, atrazine, azoxystrobin, benodanil, benoxacor, bitertanol, boscalid, buprofezin, butylate, chlorfluazuron, cumyluron, cyanazine, cycluron, cyproconazole, cyromazine, diclobutrazole, diflufenican, dimepiperate, dimethametryn, dimethenamid, dimethomorph, diniconazole, diphenamid, dipropetryn, diuron, dodemorph, epoxiconazole, ethiprole, etobenzanid, fenbuconazole, fenpropathrin, fenpropimorph, fenpyroximate, fenuron, flufenoxuron, fluometuron, flurtamone, flusilazole, flutolanil, flutriafol, forchlorfenuron, imazalil, imibenconazole, isoproturon, linuron, metconazole, methabenzthiazuron, methoprotryne, metobromuron, metoxuron, monolinuron, myclobutanil, neburon, paclobutrazole, penconazole, phenmedipham, picoxystrobin, prochloraz, prometon, prometryne, propazine, propiconazole, pymetrozine, pyraclostrobin, pyriproxyfen, sebutylazine, secbumeton, simeconazole, simetryn, tebuconazole, tebuthiuron, terbuthylazine, terbumeton, terbutryne, tetraconazole, thenylchlor, triadimefon, triadimenol, tridemorph, trifloxystrobin, triticonazole, acetochlor, alachlor, allidochlor, butachlor, butralin, chlordimeform, dazomet, dinitramine, diphenylamine, dithiopyr, fenoxanil, florasulam, flumetsulam, haloxyfop-2-ethoxyethyl, hexazinone, isopropalin, isoprothiolane, isoxaben, metazachlor, metolachlor, metosulam, metamitron, norflurazon, ofurace, picolinafen, pretilachlor, propachlor, propanil, propisochlor, prosulfocarb, pyrifenox, pyroquinolone, tepraloxydim, thiazopyr, tralkoxydim, allethrin, anilofos, aspon, bromfenvinfos, cadusafos, chlorpyrifos, coumaphos, crufomate, cycloate, cyphenothrin, defoliant, demeton-S-methyl, demeton-S-methylsulphone, dichlorvos, ditalimfos, eradicane, ethion, etrimfos, famphur, fenamiphossulfone, fenamiphos metabolite, fensulfothion, fenthion, fenthionsulfone, fenthionsulfoxide, heptenephos, iprobenfos, isazofos, isocarbamid, malathion, molinate, pebulate, phenthoate, phorate, phosalone, phosphamidon, phoxim, piperophos, pirimiphos-methyl, profenofos, propetamphos, pyraclofos, pyrazophos, pyridaphenthion, quinalphos, resmethrin, tebupirimfos, terbufos, terbufos sulfone, tetrachlorvinphos, tolclofos-methyl, triazophos, trietazine, vernolate, chloridazon, flumiclorac-pentyl, imazamethabenz-methyl, benzothiadiazole, butocarboxim, piperonyl-butoxide, propham, fludioxonil, hexaflumuron, fluorourea, cyclanilide, nitralin, terbacil, chlorthiophos, cypermethrin, demeton, phorate sulfoxide, thiometon, imazaquin, chlorpyrifos-methyl, benzoylprop, thifensulfuron-methyl, chlortoluron, trimethacarb, clomazone, bromuconazole (cis), mecarbam, propaquizafop, aldicarb, aldicarb sulfoxide, atratone, deethylatrazine, azinphos-ethyl, aziprotryn, benzofenap, bioallethrin, bioresmethrin, sulprofos, bromacil, brompyrazon, chlorimuron, cycocel, chlorpoxime, chlorsulfuron, cinidon-ethyl, cinosulfuron, coumatetralyl, cyclosulfamuron, cythioate, diethyltoluamide, desmetryne, dialifos, diallate, dichlofenthion, dichlofluanid, dichlorobenzamide, dicyclohexyl phthalate, dimefox, dimethirimol, dimethyl phthalate, dioxabenzofos, disulfoton-sulfone, dimethylbenzenesulfonamide, dodine, emamectin benzoate-Bla, emamectin benzoate-B1b, etaconazole, ethiofencarb-sulfoxide, ethoprophos, ethoxysulfuron, ethylene thiourea, ofurace, fentrazamide, flamprop-isopropyl, flamprop-methyl, fluazifop-butyl, fluazuron, flubenzimine, flurochloridone, fluridone, fluthiacet, fonofos, furmecyclox, azinphos-methyl, pyrflumetocloprid, imazapic, imazethapyr, isofenphos, isomethiozin, isouron, kadethrin, kresoxim-methyl, malaoxon, mefenpyr-diethyl, mephosfolan, mefenoxam, methfuroxam, paraoxon-methyl, monalide, monuron, naptalam, nicotine, oxycarboxin, oxydemeton-methyl, phorate-sulfone, phosfolan, pirimiphos-ethyl, propyzamide, propylenethiourea, pyrazosulfuron methyl, pyrethrin 1, pyributicarb, pyriftalid, pyrimidifen, pyrithiobac-sodium, quinoclamine, quizalofop, rabenzazole, simeton, pleocidin A, pleocidin D, tabatrex, sulfotepp, tau-fluvalinate, tetraethyl diphosphate, terbucarb, thiofanox sulfoxide, thionazin, thiophanate, triallate, triazoxide, tri-n-butyl phosphate, tri-iso-butyl phosphate, uniconazole, vamidothion sulfone and/or sulfallate.

A flow rate of the liquid chromatography is a range of 0.35-0.45 milliliters per minute (mL/min). And a condition of gradient elution is as follows: an initial proportion of the mobile phase A is 100%, the initial proportion of the mobile phase A gradually decreases to a first proportion of 80% within 4 minutes, the first proportion gradually decreases to a second proportion of 60% within 1.5 minutes, the second proportion gradually decreases to a third proportion of 0% within 4 minutes, following by remaining for 2.4 minutes, and then the third proportion gradually increases to the initial proportion of 100%.

The flow rate of the liquid chromatography is 0.4 mL/min.

A scanning range of the mass spectrometry includes: 100-900 mass-to-charge ratios (M/Z) in a positive ion mode, or 100-700 M/Z in a negative ion mode.

A resolution of the liquid mass spectrometry includes: 70000 in full mass spectrometry scanning, or 17500 in tandem mass spectrometry (MS/MS).

The method for screening the pesticide residues in the MFH characteristic agricultural products includes: step 1, the liquid chromatography-mass spectrometry database of pesticides is constructed. The standard solutions are prepared for the pesticides and the liquid chromatography and the mass spectrometry are used to detect the standard solutions; step 2, the sample is processed to obtain a processed sample, and the processed sample is detected to obtain a liquid chromatography-mass spectrogram of the processed sample; step 3, the liquid chromatography-mass spectrogram obtained from step 2 and the liquid chromatography-mass spectrograms in the liquid chromatography-mass spectrometry database obtained from step 1 are compared and analyzed. The advances of the method are as follows: a high-resolution rapid screening method for no less than 300 pesticide residues is established. the method further enhances anti-interference ability of the detection due to the high selectivity of high-resolution mass spectrometry and can be accurately and reliably applied to the rapid screening and confirmation of non-target compounds related to the safety of the MFH agricultural products, as well as high sensitivity and high-throughput multi residue analysis. The method has high qualitative accuracy and controls the probability of quality accidents to almost zero, providing a reliable technical support for the supervision of imported and exported the MFH agricultural products by customs.

### BRIEF DESCRIPTION OF DRAWINGS

FIG. 1 is a chromatogram of acephate standard in a full-scan mass spectrometry/data-dependent tandem mass spectrometry (MS/ddMS²) mode.

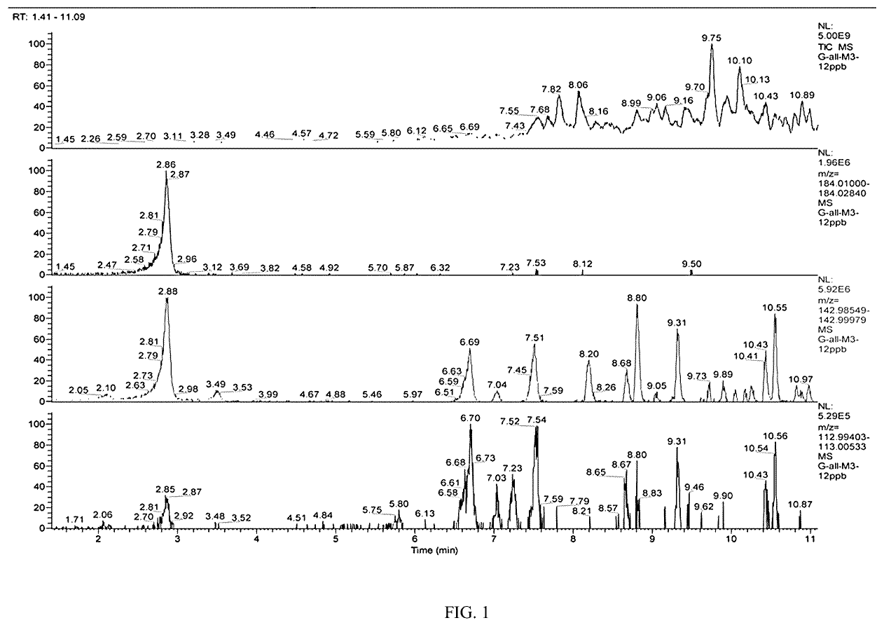

FIG. 2 is a chromatogram of acetamiprid standard in a full MS/ddMS² mode.

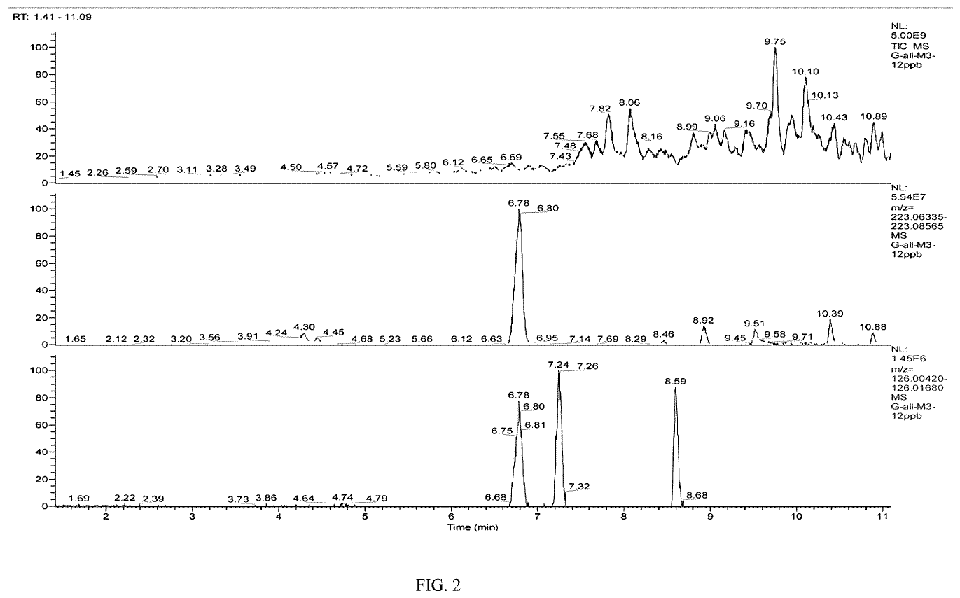

FIG. 3 is a chromatogram of aminocarb standard in a full MS/ddMS² mode.

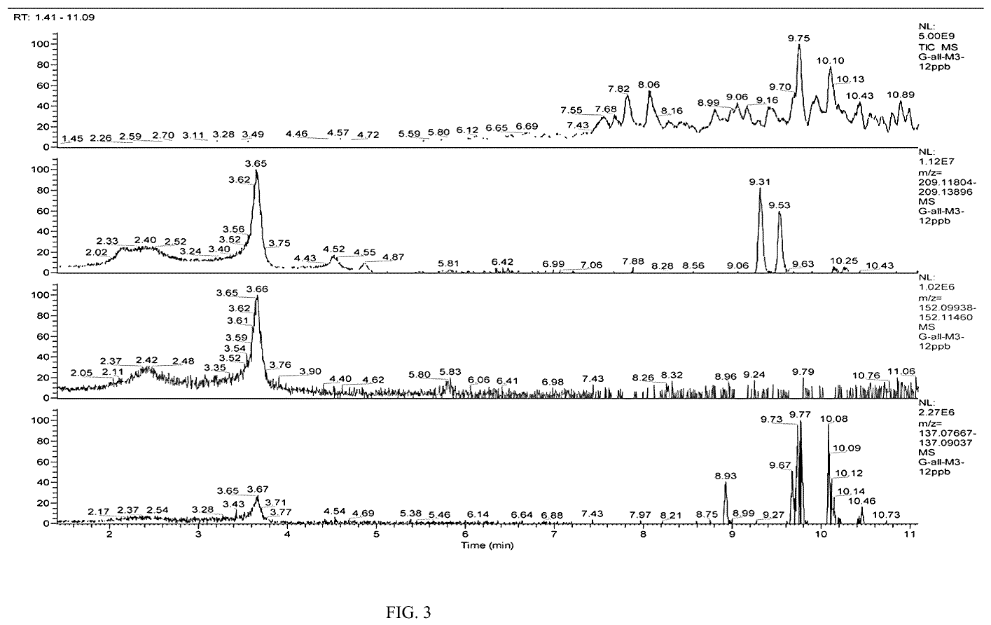

FIG. 4 is a chromatogram of azamethiphos standard in a full MS/ddMS² mode.

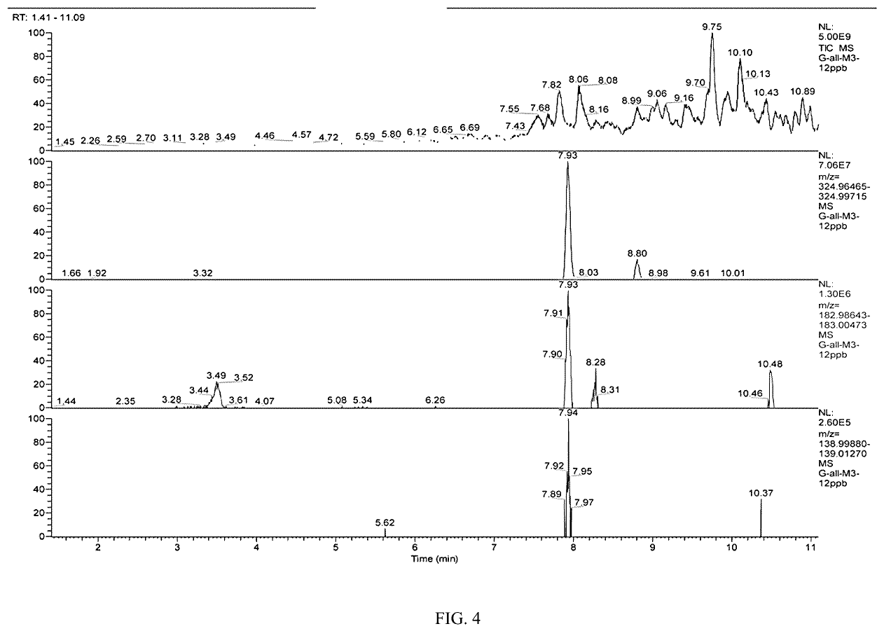

FIG. 5 is a chromatogram of benalaxyl standard in a full MS/ddMS² mode.

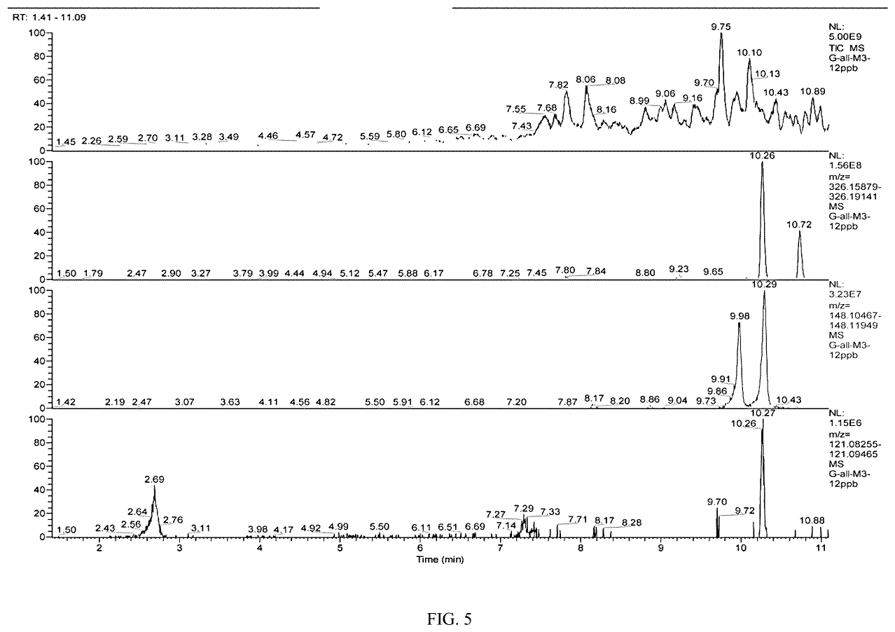

FIG. 6 is a chromatogram of bendiocarb standard in a full MS/ddMS² mode.

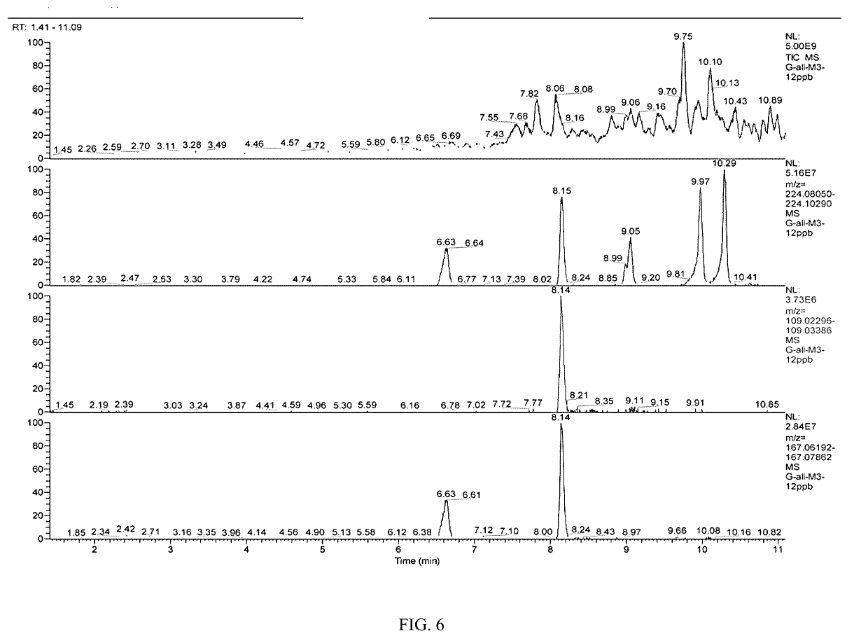

FIG. 7 is a chromatogram of benfuracarb standard in a full MS/ddMS² mode.

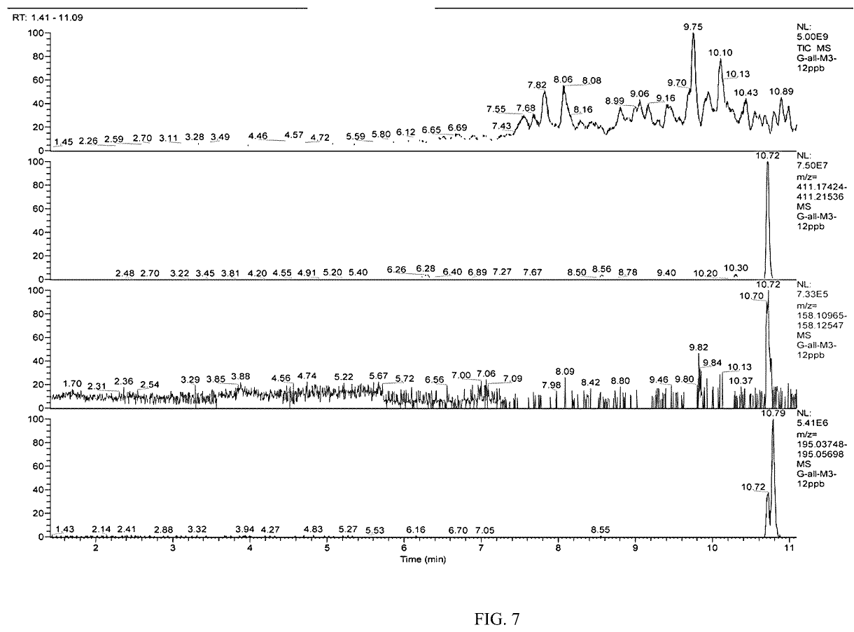

FIG. 8 is a chromatogram of bensulide standard in a full MS/ddMS² mode.

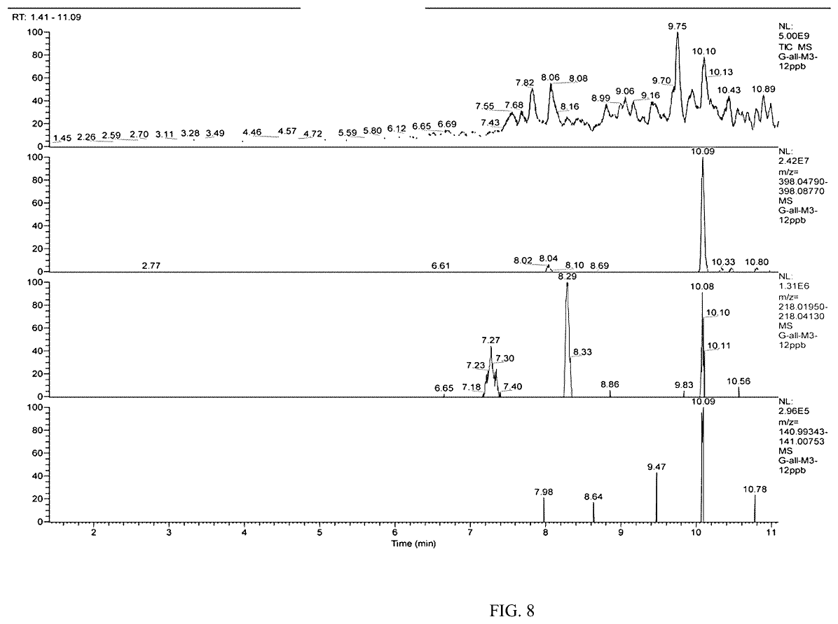

FIG. 9 is a chromatogram of benzoximate standard in a full MS/ddMS² mode.

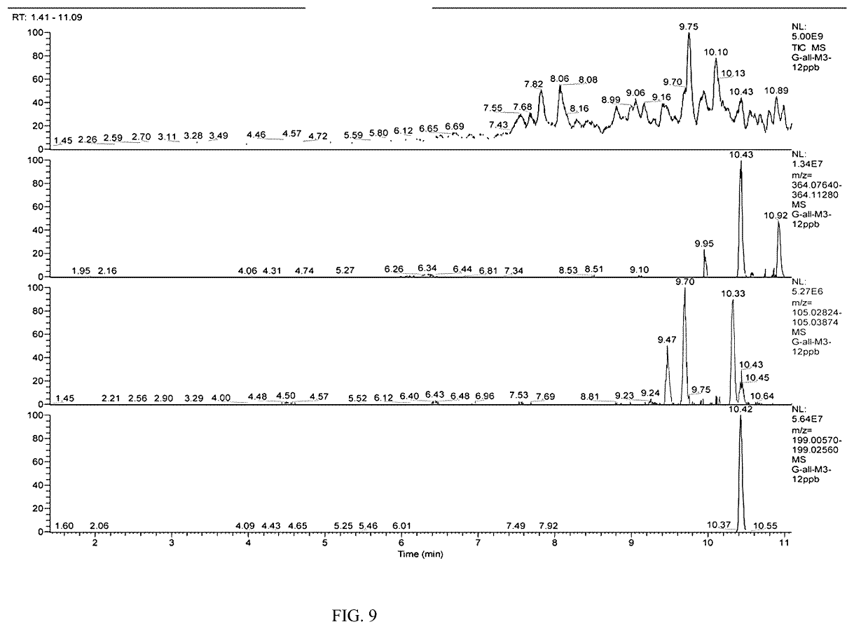

FIG. 10 is a chromatogram of bifenazate standard in a full MS/ddMS² mode.

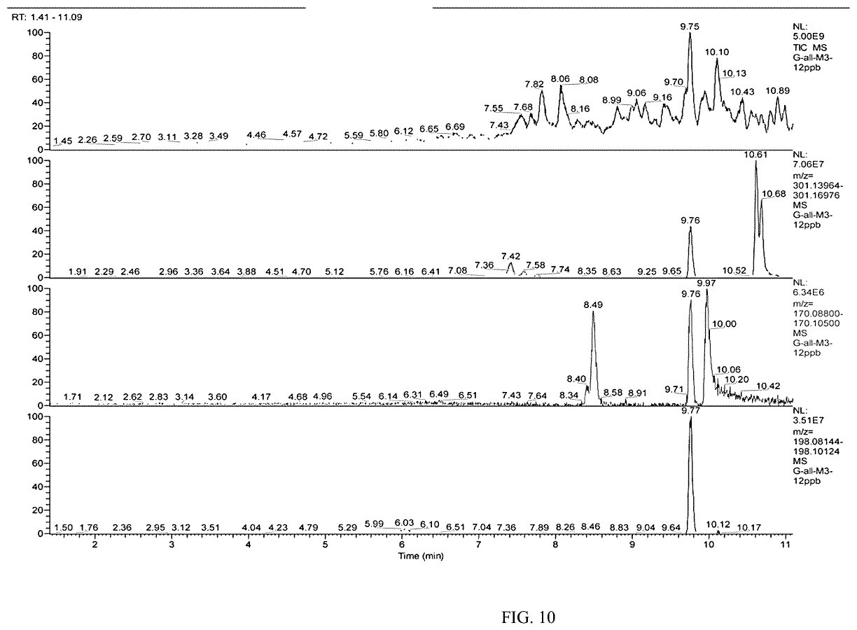

FIG. 11 is a chromatogram of bupirimate standard in a full MS/ddMS² mode.

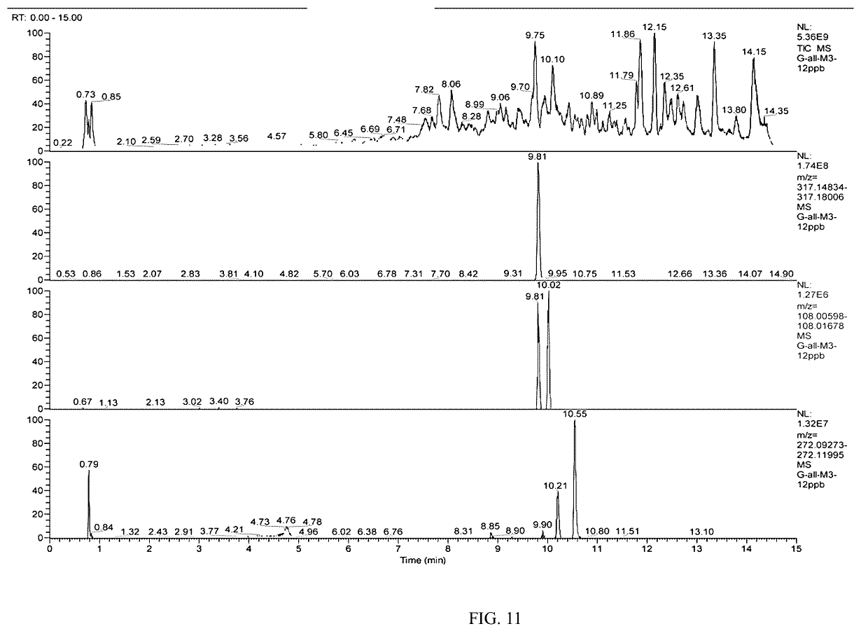

FIG. 12 is a chromatogram of butafenacil standard in a full MS/ddMS² mode.

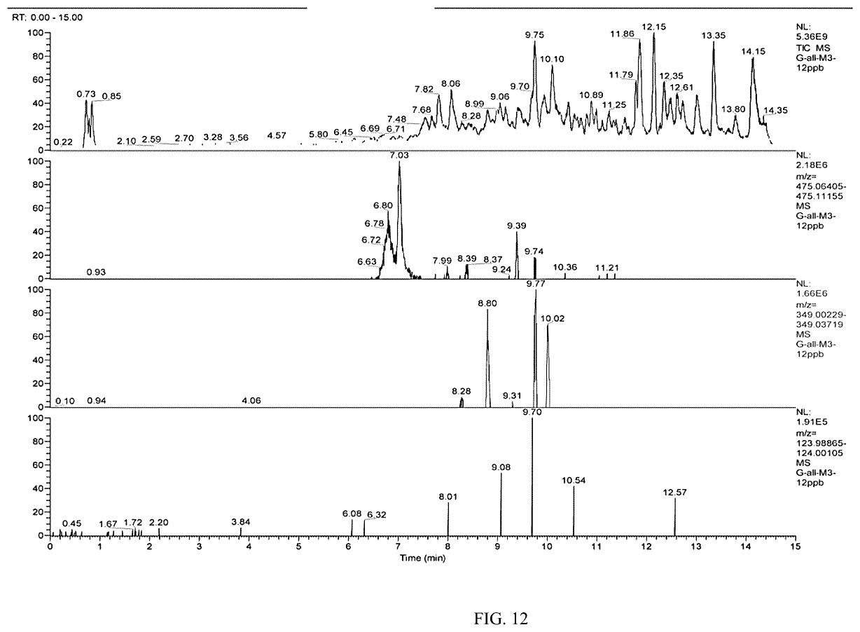

FIG. 13 is a chromatogram of butoxycarboxim standard in a full MS/ddMS² mode.

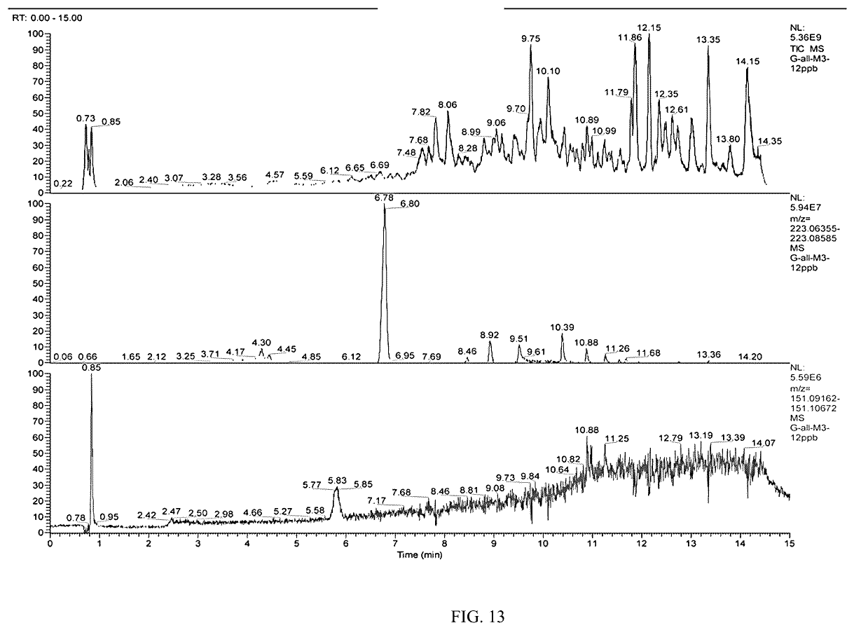

FIG. 14 is a chromatogram of buturon standard in a full MS/ddMS² mode.

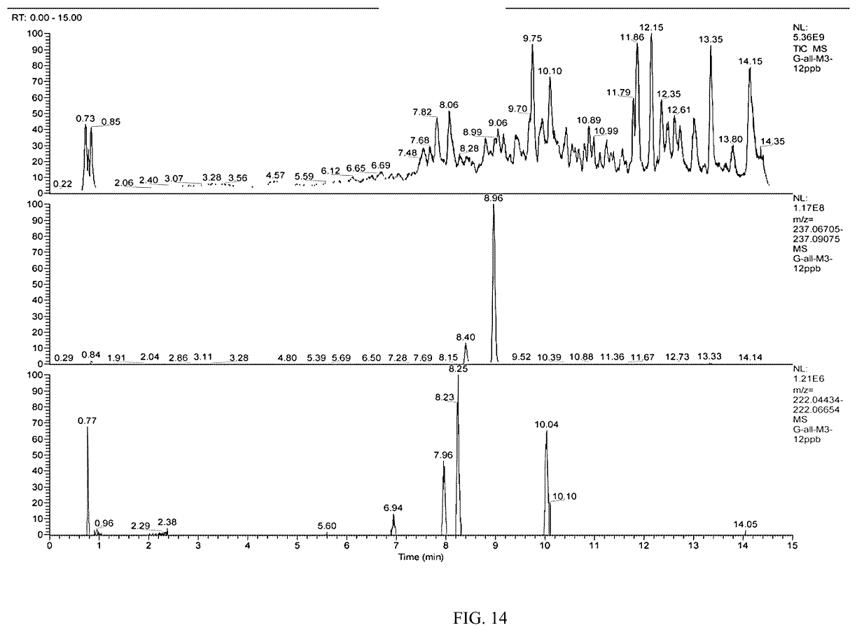

FIG. 15 is a chromatogram of carpropamid standard in a full MS/ddMS² mode.

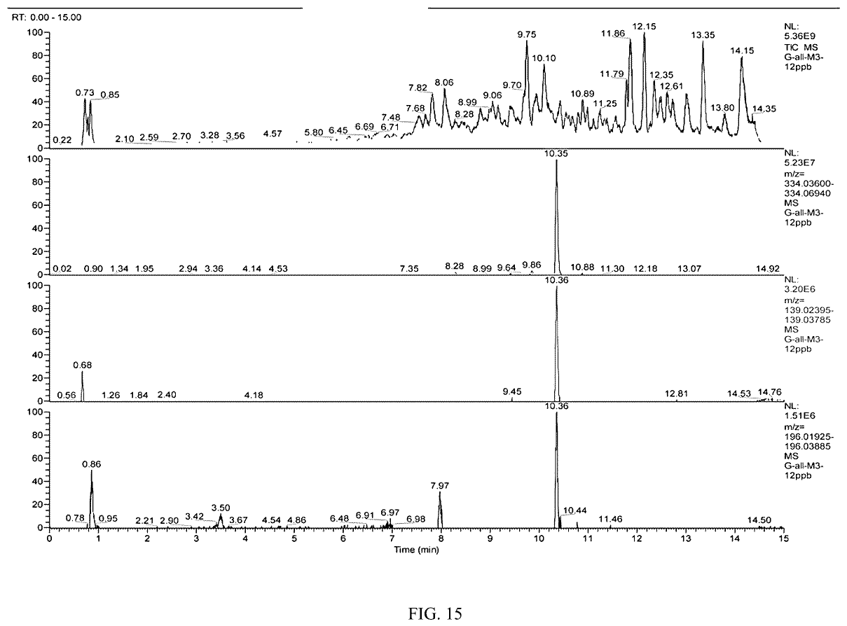

FIG. 16 is a chromatogram of carbaryl standard in a full MS/ddMS² mode.

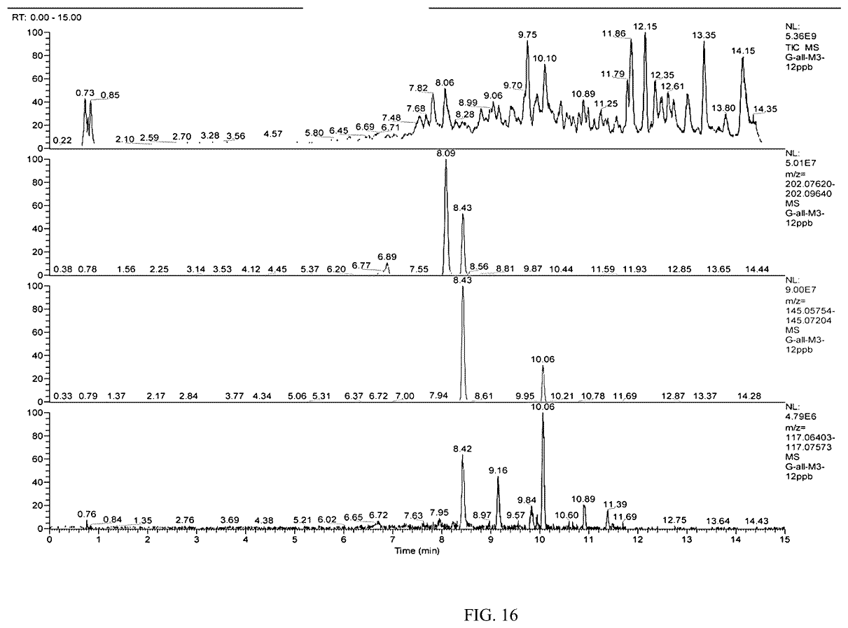

FIG. 17 is a chromatogram of carbendazim standard in a full MS/ddMS² mode.

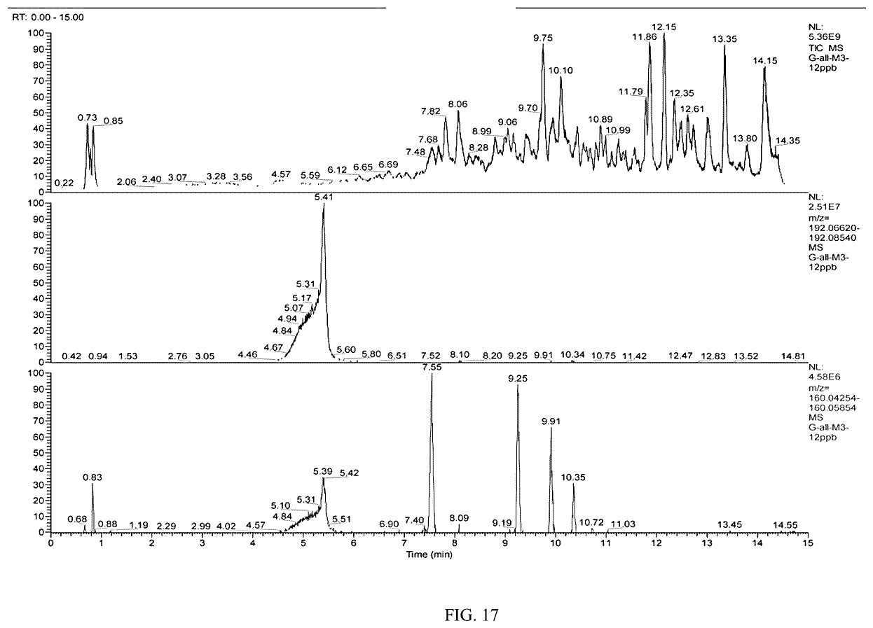

FIG. 18 is a chromatogram of carbetamide standard in a full MS/ddMS² mode.

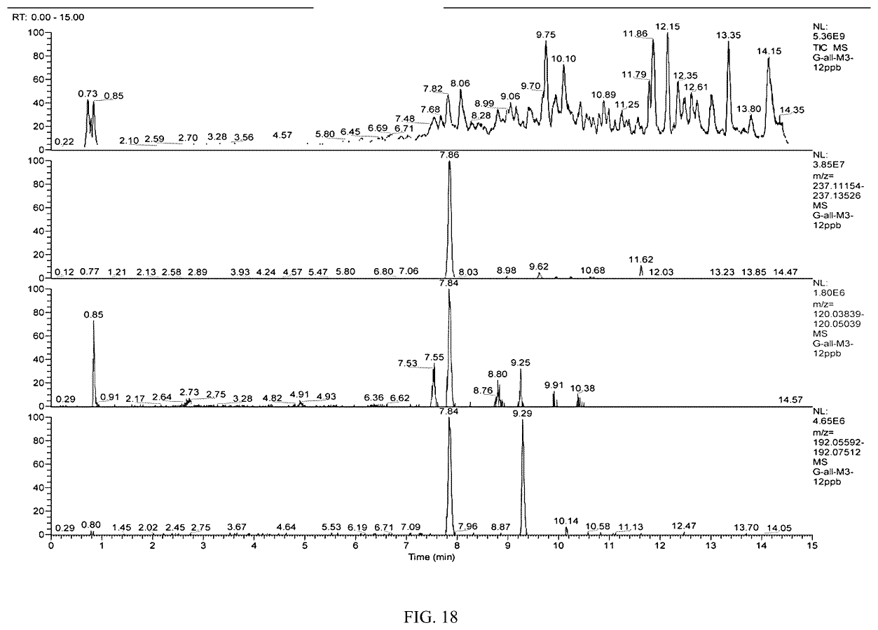

FIG. 19 is a chromatogram of carbofuran standard in a full MS/ddMS² mode.

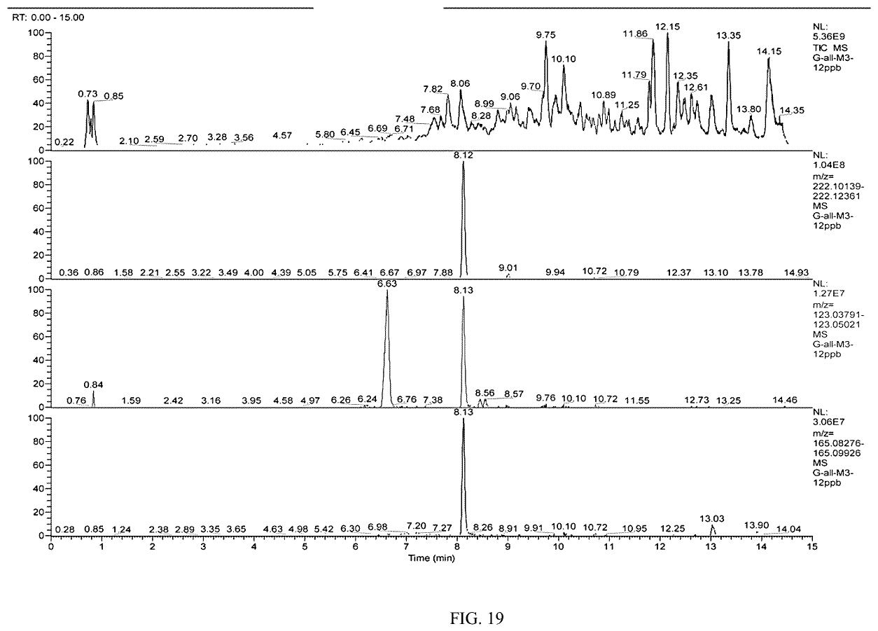

FIG. 20 is a chromatogram of carboxin standard in a full MS/ddMS² mode.

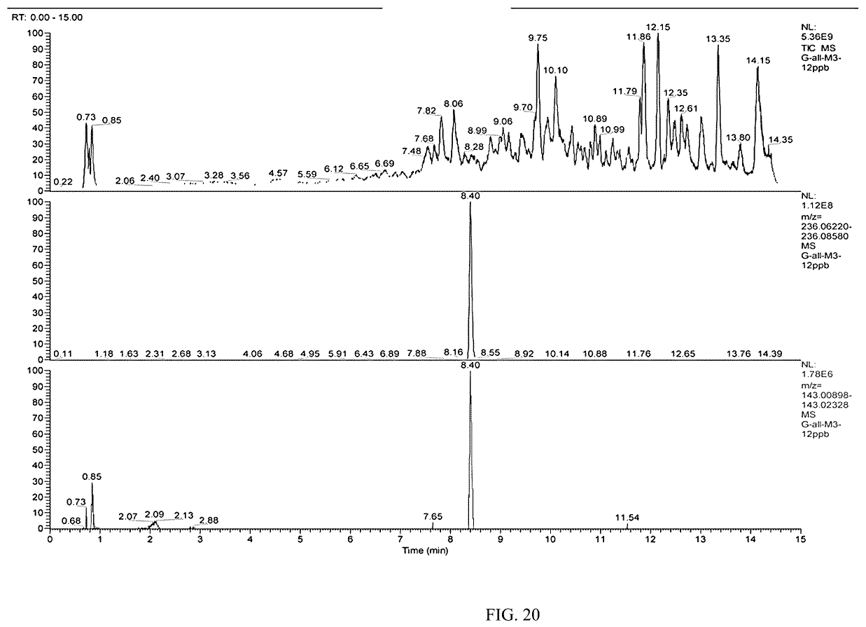

FIG. 21 is a chromatogram of clethodim standard in a full MS/ddMS² mode.

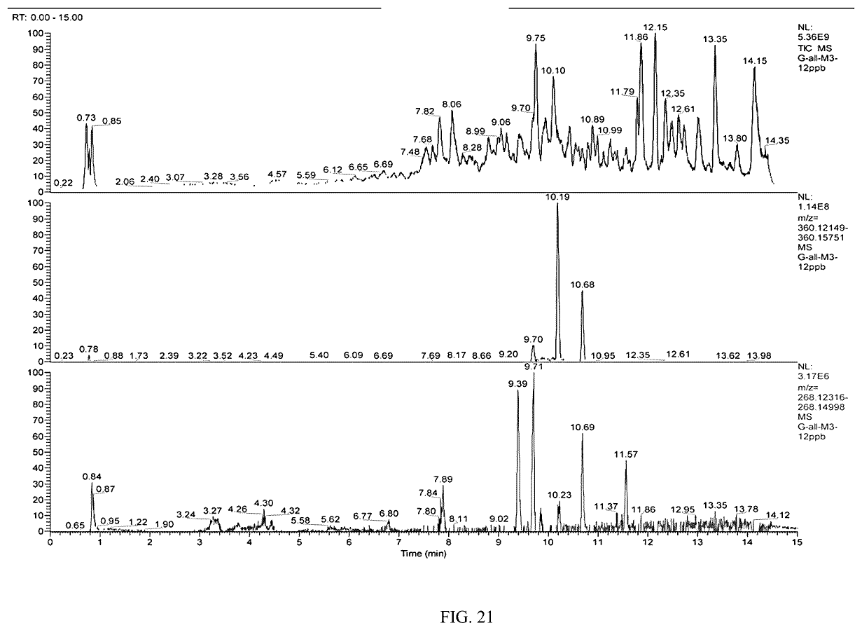

FIG. 22 is a chromatogram of clothianidin standard in a full MS/ddMS² mode.

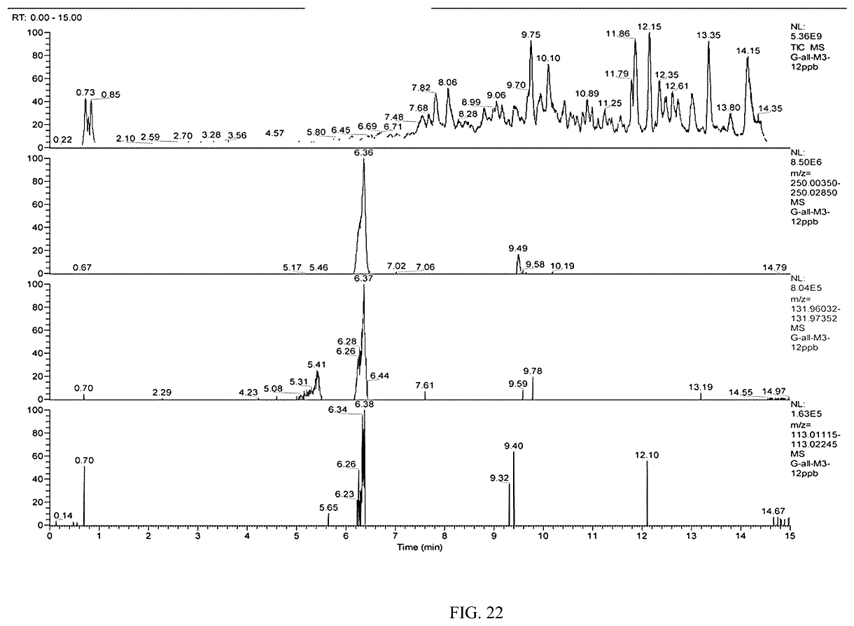

FIG. 23 is a chromatogram of cyazofamid standard in a full MS/ddMS² mode.

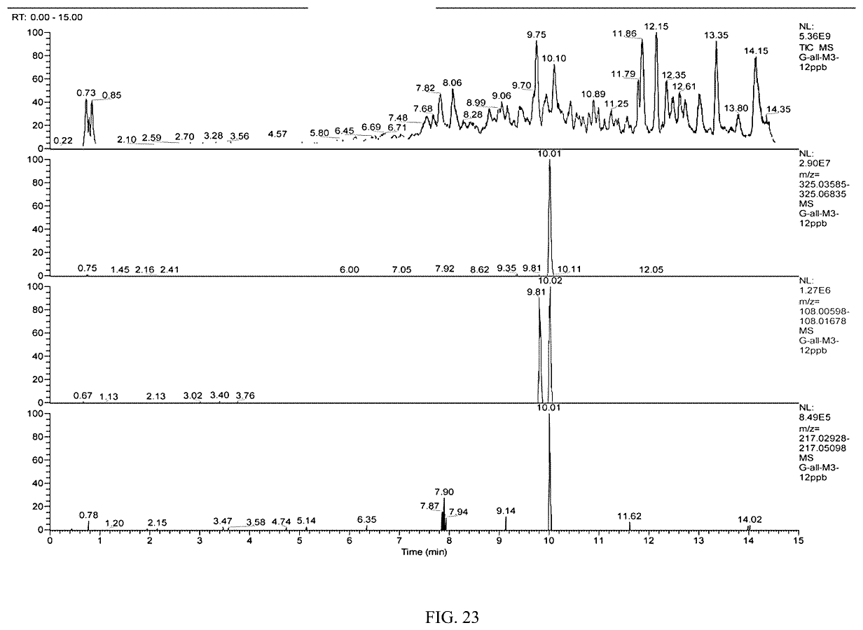

FIG. 24 is a chromatogram of cycloxydim standard in a full MS/ddMS² mode.

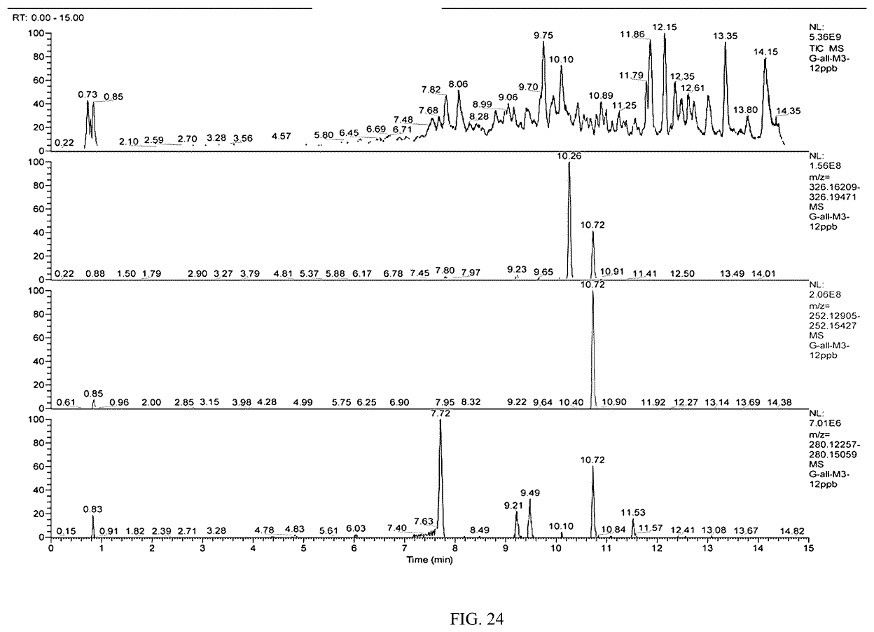

FIG. 25 is a chromatogram of cyprodinil standard in a full MS/ddMS² mode.

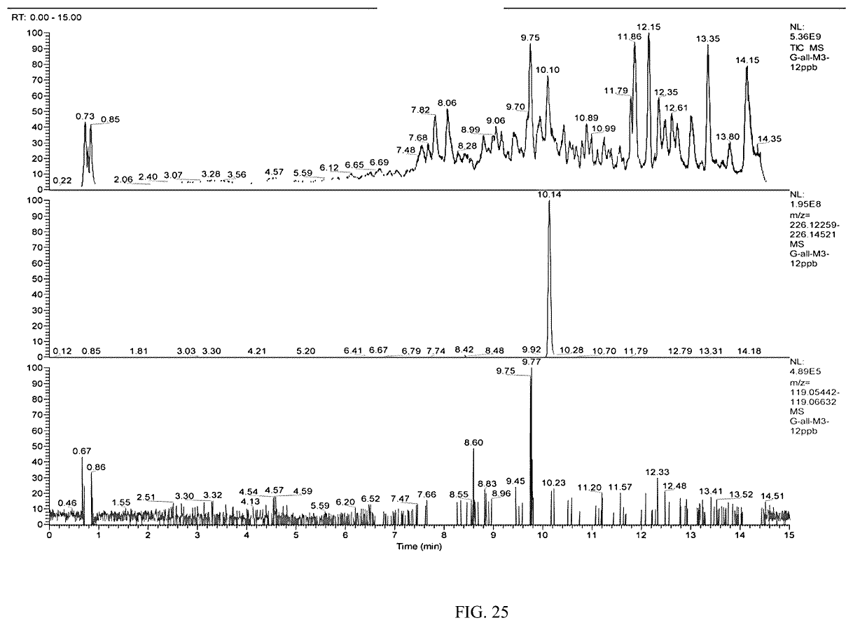

FIG. 26 is a chromatogram of desmedipham standard in a full MS/ddMS² mode.

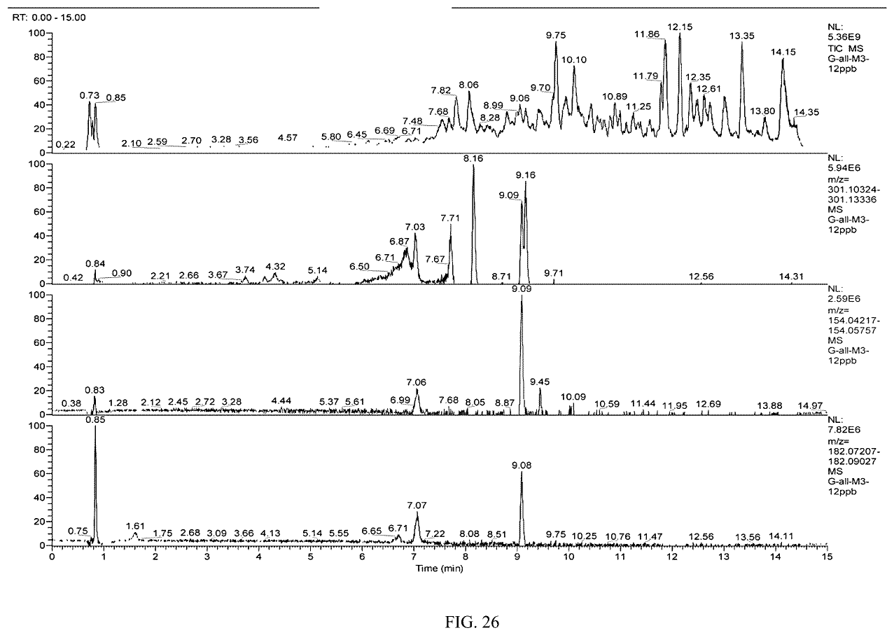

FIG. 27 is a chromatogram of dicrotophos standard in a full MS/ddMS² mode.

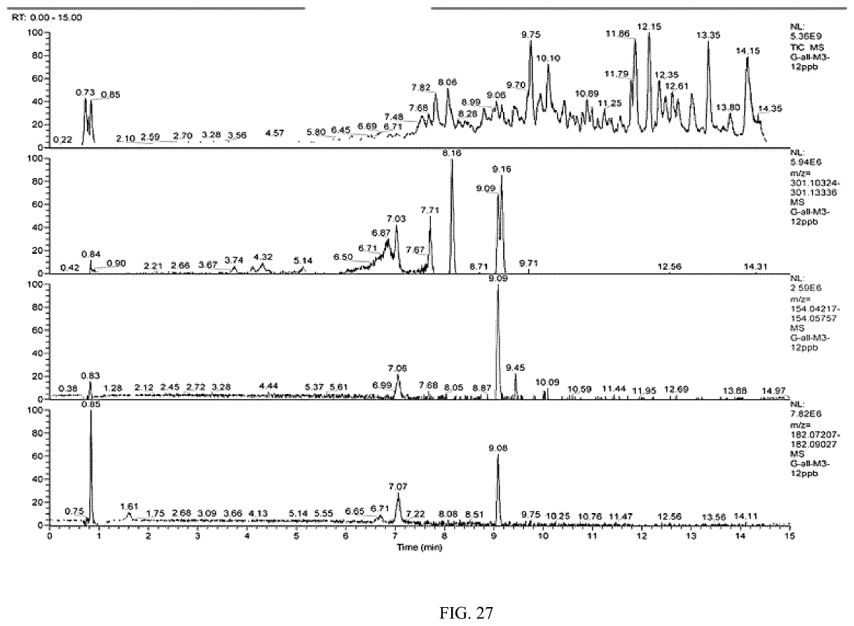

FIG. 28 is a chromatogram of diethofencarb standard in a full MS/ddMS² mode.

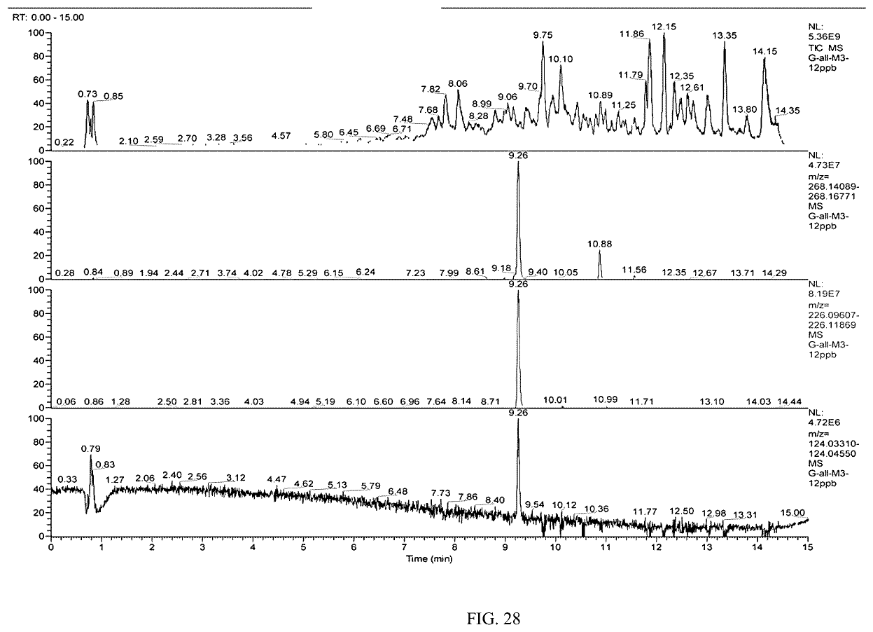

FIG. 29 is a chromatogram of dimethoate standard in a full MS/ddMS² mode.

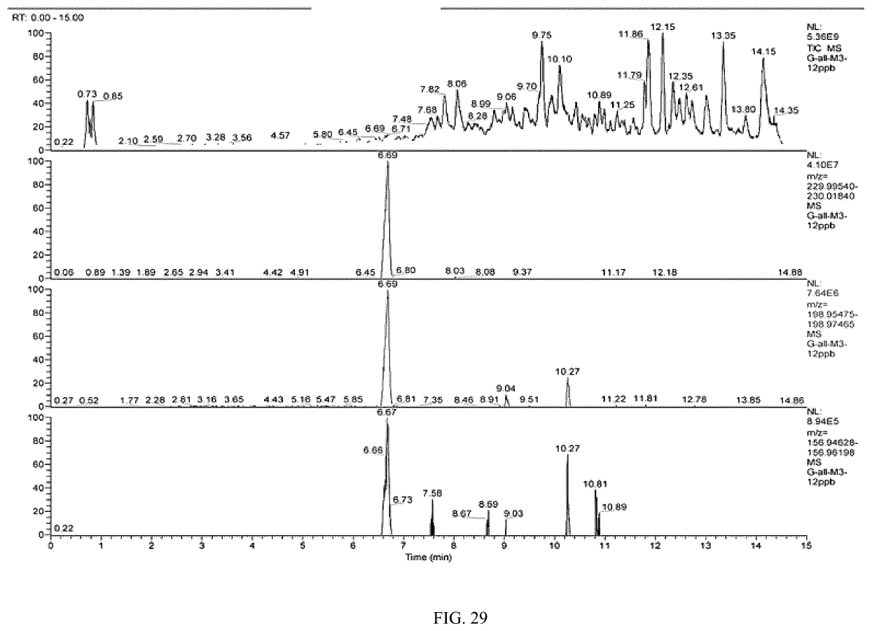

FIG. 30 is a chromatogram of dinotefuran standard in a full MS/ddMS² mode.

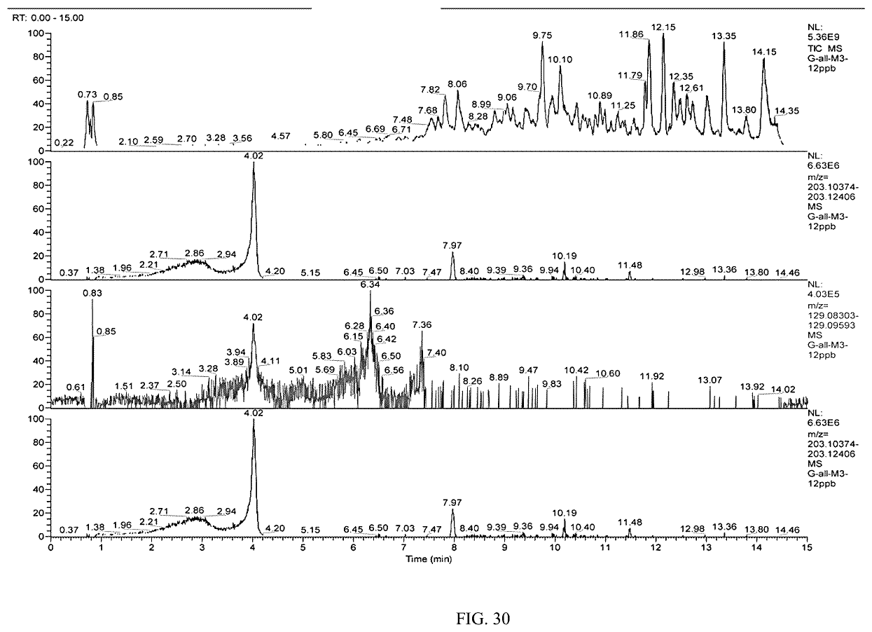

### DETAILED DESCRIPTION OF EMBODIMENTS

### Embodiment 1

A database is constructed for simultaneously screening multiple pesticide residues.

1. Preparation and Preservation of Samples

(1) Preparation of Samples

An edible part of a detecting object is chopped, then mixed evenly and sealed to use as a sample and followed by marking the sample to obtain a marked sample. The marked sample is placed at 0° C.-4° C. for cold storage.

(2) Pre-Processing Steps

The marked sample weighed to 5 grams (g) (accurated to 0.01 g) is put into a 50 mL plastic centrifuge tube, and acetonitrile of 10 mL, magnesium sulfate of 4 g, sodium chloride of 1 g, sodium citrate of 1 g, disodium hydrogen citrate of 0.5 g, and a ceramic homoproton are added into the 50 mL plastic centrifuge tube to obtain a first mixture, then the 50 mL plastic centrifuge tube is covered with a cover and shook vigorously for 1 minute (min), followed by centrifuging the first mixture at 4200 r/min for 5 min to obtain a first mixed solution. A supernatant of the first mixed solution of 6 mL is taken and added into a 15 mL plastic centrifuge tube containing 900 mg of magnesium sulfate and 150 mg of primary secondary amine (PSA) to obtain a second mixture. For the marked sample with a darker color, the magnesium sulfate of 885 mg, PSA of 150 mg, and graphitized carbon black (GCB) of 15 mg are added to the 15 mL plastic centrifuge tube, followed by vortex mixing for 1 min. The second mixture is centrifuged at 4200 r/min for 5 minutes to obtain a second mixed solution. A supernatant of the second mixed solution of 2 mL is accurately pipetted into a 10 mL test tube, and blown in a 40° C. water bath with nitrogen until almost dry. Acetonitrilxe and water (1:1) are added into the test tube to reconstitute, then filtered through a microporous membrane to obtain a filtered solution for detection.

2. Detection

(1) A Condition of Liquid Chromatography
a. A liquid chromatography column: an Accucore™ aQ C18 hydrophilic interaction liquid chromatography (HPLC) column, 150 millimeter (mm)×2.1 mm (i.d.), a particle size of 2.6 micrometer (μm), or equivalents;
b. A gradient elution procedure of the mobile phases is shown in Table 1;
c. The mobile phases: aqueous phase A (W) of ammonium formate of 5 millimol per liter (mM) and a 0.1% formic acid aqueous solution, and organic phase B (M) of ammonium formate of 5 mM and a 0.1% formic acid methanol solution;
d. A flow velocity: 0.4 mL/min;
e. A sample volume: 0.5 μL;
f. A column temperature: 25° C.

TABLE 1
The gradient elution procedure of the mobile phases

| time/min | aqueous phase % | organic phase % |
|---|---|---|
| 0 | 100 | 0 |
| 4 | 80 | 20 |
| 5.5 | 60 | 40 |
| 9.5 | 0 | 100 |
| 11.9 | 0 | 100 |
| 15 | 100 | 0 |

(2) A Condition of Mass Spectrometry

An ion source: an electrospray ion source with heated electrospray ionization (HESI-II) probe; a temperature of the ion source: 325° C.; a temperature of a heater: 350° C.; a sheath gas flow rate: 40 atmospheres relative to vacuum (arb); an aux gas flow rate: 10 arb; a scanning range of the mass spectrometry: 100-900 mass-to-charge ratios (MIZ) in a positive ion mode or 100-700 M/JZ in a negative ion mode; a resolution of the mass spectrometry: 70000 in full mass spectrometry scanning, or 17500 in tandem mass spectrometry (MS/MS); names of the pesticides, molecular formulas of the pesticides, accurate masses of the pesticides and accurate masses of characteristic ion fragments are shown in Table 2.

TABLE 2
Names of the pesticides, molecular formulas of the pesticides, accurate masses of the pesticides and accurate masses of characteristic ion fragments

| | Molecular | Retention | Scan | Parent | Characteristic ion fragments | | | | |
|---|---|---|---|---|---|---|---|---|---|
| Name | formulas | times | mode | ion | 1 | 2 | 3 | 4 | 5 |
| Acephate | C₄H₁₀NO₃PS | 2.76 | Positive | 184.0192 | 142.99264 | 112.99968 | | | |
| Acetamiprid | C₁₀H₁₁ClN₄ | 6.71 | Positive | 223.0745 | 126.0105 | 98.9997 | 90.03383 | 72.98408 | 56.04948 |
| Aminocarb | C₁₁H₁₆N₂O₂ | 3.57 | Positive | 209.1285 | 152.10699 | 137.08352 | | | |
| Azamethiphos | C₉H₁₀ClN₂O₅PS | 7.86 | Positive | 324.9809 | 182.99558 | 127.98977 | 139.00575 | | |
| Benalaxyl | C₂₀H₂₃NO₃ | 10.19 | Positive | 326.1751 | 148.11208 | 208.13321 | 91.05423 | 121.0886 | 266.15394 |
| Bendiocarb | C₁₁H₁₃NO₄ | 8.07 | Positive | 224.0917 | 109.02841 | 167.07027 | 127.03897 | 123.044 | 165.091 |
| Benfuracarb | C₂₀H₃₀N₂O₅S | 10.65 | Positive | 411.1948 | 158.11756 | 195.04723 | 252.06905 | 148.04268 | 90.0372 |
| Bensulide | C₁₄H₂₄NO₄PS₃ | 10.02 | Positive | 398.0678 | 218.0304 | 141.00048 | 200.01989 | 61.01065 | 158.02703 |
| Benzoximate | C₁₈H₁₈ClNO₅ | 10.35 | Positive | 364.0946 | 105.03349 | 199.01565 | 95.04914 | 183.99217 | 140.97378 |
| Bifenazate | C₁₇H₂₀N₂O₃ | 9.68 | Positive | 301.1547 | 170.0965 | 198.09134 | 148.07569 | 120.08078 | 91.05423 |
| Bupirimate | C₁₃H₂₄N₄O₃S | 9.75 | Positive | 317.1642 | 108.01138 | 166.09749 | 272.10634 | 150.10257 | 208.14444 |
| Butafenacil | C₂₀H₁₈ClF₃N₂O₆ | 9.7 | Positive | 475.0878 | 179.98468 | 331.00918 | 123.99485 | 349.01974 | |
| Butoxycarboxim | C₇H₁₄N₂O₄S | 6.71 | Positive | 223.0747 | 167.03726 | 181.05291 | 90.03383 | 151.09917 | |
| Buturon | C₁₂H₁₃ClN₂O | 8.9 | Positive | 237.0789 | 84.08078 | 126.0105 | 222.05544 | 128.02611 | 167.03705 |
| Carpropamid | C₁₅H₁₈ClNO | 10.29 | Positive | 334.0527 | 139.0309 | 196.02905 | 160.05237 | 103.05423 | |
| Carbaryl | C₁₂H₁₁NO₂ | 8.36 | Positive | 202.0863 | 145.06479 | 117.06988 | 155.06061 | 127.05423 | 115.05423 |
| Carbendazim | C₉H₉N₃O₂ | 5.32 | Positive | 192.0768 | 160.05054 | 132.05562 | 105.04472 | 92.04948 | |
| Carbetamide | C₁₂H₁₆N₂O₃ | 7.78 | Positive | 237.1234 | 118.08626 | 120.04439 | 192.06552 | 100.07569 | 72.08078 |
| Carbofuran | C₁₂H₁₅NO₃ | 8.05 | Positive | 222.1125 | 123.04406 | 165.09101 | 137.05971 | 147.08044 | |
| Carboxin | C₁₂H₁₃NO₂S | 8.33 | Positive | 236.074 | 143.01613 | 93.0573 | | | |
| Clethodim | C₁₇H₂₆ClNO₃S | 10.62 | Positive | 360.1395 | 164.0706 | 268.13657 | 206.11755 | 136.07569 | 240.1053 |
| Clothianidin | C₆H₈ClN₅O₂S | 6.29 | Positive | 250.016 | 131.96692 | 113.0168 | 169.05441 | 110.07127 | |
| Cyazofamid | C₁₃H₁₃ClN₄O₂S | 9.95 | Positive | 325.0521 | 108.01138 | 216.0323 | 217.04013 | | |
| Cycloxydim | C₁₇H₂₇NO₃S | 10.67 | Positive | 326.1784 | 180.10191 | 101.04195 | 155.05251 | 252.14166 | 280.13658 |
| Cyprodinil | C₁₄H₁₅N₃ | 10.07 | Positive | 226.1339 | 93.0573 | 91.05423 | 108.08708 | 118.05255 | 119.06037 |
| Desmedipham | C₁₆H₁₆N₂O₄ | 9.02 | Positive | 301.1183 | 136.0393 | 154.04987 | 182.08117 | 108.04439 | |
| Dicrotophos | C₈H₁₆NO₅P | 6.02 | Positive | 238.0839 | 112.07569 | 127.01547 | 72.04439 | 193.02604 | |
| Diethofencarb | C₁₄H₂₁NO₄ | 9.19 | Positive | 268.1543 | 180.10191 | 152.0706 | 226.10738 | 124.0393 | 198.07608 |
| Dimethoate | C₅H₁₂NO₃PS₂ | 6.62 | Positive | 230.0069 | 198.9647 | 170.98978 | 88.02155 | 142.99286 | 156.95413 |
| Dinotefuran | C₇H₁₄N₄O₃ | 3.92 | Positive | 203.1139 | 129.08948 | 114.10231 | 113.09456 | 157.12086 | |
| Dioxacarb | C₁₁H₁₃NO₄ | 6.57 | Positive | 224.0917 | 123.04406 | 95.04914 | 167.07027 | 162.05495 | |
| Ethiofencarb | C₁₁H₁₅NO₂S | 8.48 | Positive | 226.0896 | 107.04914 | 164.07059 | 95.04914 | 79.05423 | |
| Ethirimol | C₁₁H₁₉N₃O | 7.32 | Positive | 210.1601 | 140.10699 | 98.06004 | 182.12879 | 193.13354 | 70.06513 |
| Ethofumesate | C₁₃H₁₈O₅S | 9.6 | Positive | 287.0948 | 121.06479 | 259.06347 | 241.05291 | 162.06753 | 161.05971 |
| Fenazaquin | C₂₀H₂₂N₂O | 11.5 | Positive | 307.1805 | 161.13248 | 147.05529 | 146.109 | 105.06988 | 131.08553 |
| Fenhexamid | C₁₄H₁₇Cl₂NO₂ | 9.71 | Positive | 302.0709 | 170.09643 | 198.09134 | 153.06988 | 184.07569 | 97.10118 |
| Fenobucarb | C₁₂H₁₇NO₂ | 9.24 | Positive | 208.1332 | 95.04914 | 152.0706 | 151.11174 | 57.06988 | |
| Fenoxycarb | C₁₇H₁₉NO₄ | 10.03 | Positive | 302.1387 | 88.0393 | 116.0706 | 256.09682 | | |
| Fenpropidin | C₁₉H₃₁N | 9.1 | Positive | 274.2529 | 147.11683 | 86.09643 | 117.06988 | 132.09335 | 119.08553 |
| Flufenacet | C₁₄H₁₃F₄N₃O₂S | 9.82 | Positive | 364.0737 | 152.05062 | 194.09757 | 124.0557 | | |
| Fuberidazole | C₁₁H₈N₂O | 6.41 | Positive | 185.0709 | 157.07602 | 156.0682 | 92.04948 | 118.05255 | |
| Furalaxyl | C₁₇H₁₉NO₄ | 9.2 | Positive | 302.1387 | 242.11756 | 95.01276 | 270.11247 | | |
| Furathiocarb | C₁₈H₂₆N₂O₅S | 10.72 | Positive | 383.1635 | 195.0474268 | 252.06889 | 164.08318 | | |
| Hexythiazox | C₁₇H₂₁ClN₂O₂S | 11 | Positive | 353.1085 | 228.02444 | 168.05745 | 194.03672 | 271.03025 | 151.0309 |
| Hydramethylnon | C₂₅H₂₄F₆N₄ | 10.49 | Positive | 495.1978 | 323.14781 | 368.08739 | 171.04161 | 238.05866 | 128.11822 |
| Imidacloprid | C₉H₁₀ClN₅O₂ | 6.23 | Positive | 256.0596 | 209.05885 | 175.09793 | 84.05562 | 210.06668 | |
| Indoxacarb | C₂₂H₁₇ClF₃N₃O₇ | 10.47 | Positive | 528.078 | 218.04234 | 203.01886 | 190.00542 | 168.02116 | 249.0426 |
| Isoprocarb | C₁₁H₁₅NO₂ | 8.72 | Positive | 194.1176 | 95.04914 | 137.09609 | 152.0706 | | |
| Mefenacet | C₁₆H₁₄N₂O₂S | 9.64 | Positive | 299.0849 | 148.07569 | 120.08078 | 192.01138 | 91.05423 | 136.02155 |
| Mepanipyrim | C₁₄H₁₃N₃ | 9.84 | Positive | 224.1182 | 106.06513 | 131.06037 | 79.05423 | 105.04472 | 104.04948 |
| Mepronil | C₁₇H₁₉NO₂ | 9.57 | Positive | 270.1489 | 119.04914 | 228.10191 | 136.0393 | 91.05423 | 93.06988 |
| Metalaxyl | C₁₅H₂₁NO₄ | 8.78 | Positive | 280.1543 | 220.13321 | 192.13829 | 160.11208 | 248.12812 | 148.11208 |
| Methamidophos | C₂H₈NO₂PS | 2 | Positive | 142.0086 | 124.98206 | 109.98229 | 94.00524 | | |
| Methomyl | C₅H₁₀N₂O₂S | 4.83 | Positive | 163.0536 | 106.03211 | 88.02155 | | | |
| Methoxyfenozide | C₂₂H₂₈N₂O₃ | 9.59 | Positive | 369.2173 | 149.05971 | 133.06479 | 91.05423 | 109.06479 | 105.06988 |
| Metolcarb | C₉H₁₁NO₂ | 7.71 | Positive | 166.0863 | 109.06479 | 94.04101 | 91.05423 | 81.06988 | |
| Metribuzin | C₈H₁₄N₄OS | 7.97 | Positive | 215.0961 | 187.10119 | 131.03859 | 84.08078 | 89.0168 | 74.0059 |
| Napropamide | C₁₇H₂₁NO₂ | 9.84 | Positive | 272.1645 | 129.11483 | 171.08044 | 199.07536 | 114.09134 | 74.09643 |
| Nitenpyram | C₁₁H₁₅ClN₄O₂ | 4.65 | Positive | 271.0956 | 224.0949 | 196.0636 | 126.0105 | 99.09167 | 210.07925 |
| Nuarimol | C₁₇H₁₂ClFN₂O | 9.29 | Positive | 315.0695 | 81.04476 | 252.08891 | 243.0372 | 138.99452 | 123.02407 |
| Omethoate | C₅H₁₂NO₄PS | 3.39 | Positive | 214.0297 | 142.99263 | 182.98754 | 127.01547 | 154.99263 | 196.01918 |
| (Dimethoateoxon) | | | | | | | | | |
| Oxamyl | C₇H₁₃N₃O₃S | 4.52 | Positive | 220.075 | 72.04439 | 90.05495 | | | |
| Pencycuron | C₁₉H₂₁ClN₂O | 10.46 | Positive | 329.1415 | 125.01525 | 261.07892 | 210.1044 | | |
| Pirimicarb | C₁₁H₁₈N₄O₂ | 7.29 | Positive | 239.1503 | 72.04439 | 182.12895 | 109.07602 | 137.07094 | |
| Propamocarb | C₉H₂₀N₂O₂ | 3.65 | Positive | 189.1598 | 102.05495 | 144.10191 | | | |
| Propargite | C₁₉H₂₆O₄S | 11.05 | Positive | 351.1625 | 175.11174 | 231.17434 | 107.04914 | 81.06988 | 163.11174 |
| Propoxur | C₁₁H₁₅NO₃ | 8 | Positive | 210.1125 | 111.04406 | 168.06552 | 153.09101 | 93.03349 | |
| Prosulfocarb | C₁₄H₂₁NOS | 10.67 | Positive | 252.1417 | 91.05423 | 128.10699 | 86.06004 | 65.03858 | |
| Pyrimethanil | C₁₂H₁₃N₃ | 9.16 | Positive | 200.1182 | 107.06037 | 82.06513 | 183.09167 | 168.0682 | 183.09167 |
| Quinoxyfen | C₁₅H₈CLFNO | 11.1 | Positive | 308.004 | 162.01064 | 196.97951 | 213.98221 | 272.0273 | 75.02293 |
| Rotenone | C₂₃H₂₂O₆ | 9.92 | Positive | 395.1489 | 213.09101 | 192.0781 | 203.07027 | 179.07027 | 191.07027 |
| Spirodiclofen | C₂₁H₂₄C₁₂O₄ | 11.21 | Positive | 411.1124 | 71.08553 | 313.03928 | 295.02871 | 212.95046 | 184.95555 |
| Tebufenozide | C₂₂H₂₈N₂O₂ | 10.03 | Positive | 353.2224 | 133.06479 | 105.06988 | 72.08078 | 103.05423 | 95.04919 |
| Tebutam | C₁₅H₂₃NO | 9.89 | Positive | 234.1852 | 91.05423 | 192.13829 | 142.12264 | 84.08078 | |
| Temephos | C₁₆H₂₀O₆P₂S₃ | 10.83 | Positive | 466.997 | 142.99275 | 341.00656 | 404.978 | 248.98035 | 437.0042 |
| Thiabendazole | C₁₀H₇N₃S | 6.13 | Positive | 202.0433 | 175.03245 | 131.06037 | 92.04948 | | |
| Thiacloprid | C₁₀H₉ClN₄S | 7.18 | Positive | 253.0309 | 126.0105 | 98.9996 | 90.03383 | | |
| Thiamethoxam | C₈H₁₀ClN₅O₃S | 5.33 | Positive | 292.0266 | 131.96692 | 181.05447 | 211.06481 | 152.02795 | 122.07143 |
| Thiobencarb | C₁₂H₁₆ClNOS | 10.39 | Positive | 258.0714 | 125.01525 | 100.07569 | | | |
| Thiodicarb | C₁₀H₁₈N₄O₄S₃ | 8.43 | Positive | 355.0563 | 88.0215465 | 107.99362 | 78.96707 | | |
| Thiophanate-methyl | C₁₂H₁₄N₄O₄S₂ | 8 | Positive | 343.0529 | 151.03245 | 311.02671 | 268.02089 | 194.03826 | 235.99468 |
| Trichlorfon | C₄H₈C₁₃O₄P | 6.42 | Positive | 256.9299 | 127.01552 | 220.95318 | 78.99434 | | |
| Vamidothion | C₈H₁₈NO₄PS₂ | 6.6 | Positive | 288.0488 | 118.03211 | 146.06341 | 86.06004 | 119.03657 | 104.07061 |
| Ametryn | C₉H₁₇N₅S | 8.84 | Positive | 228.1277 | 186.08079 | 96.05562 | 116.02769 | 71.06037 | 138.07742 |
| Atrazine | C₈H₁₄ClN₅ | 8.82 | Positive | 216.101 | 174.0541 | 96.05562 | 104.001 | 132.0323 | 146.0228 |
| Azoxystrobin | C₂₂H₁₇N₃O₅ | 9.28 | Positive | 404.1241 | 344.10397 | 329.07949 | 372.09788 | 328.07167 | 300.07675 |
| Benodanil | C₁₃H₁₀INO | 8.84 | Positive | 323.988 | 230.93013 | 94.04132 | 76.03075 | 104.02567 | |
| Benoxacor | C₁₁H₁₁Cl₂O₂ | 9.28 | Positive | 260.024 | 149.08352 | 120.04439 | 134.06004 | 150.09134 | 160.07569 |
| Bitertanol | C₂₀H₂₃N₃O₂ | 10.41 | Positive | 338.1863 | 70.03997 | 99.08044 | 269.15361 | 251.14304 | 183.08044 |
| Boscalid | C₁₈H₁₂Cl₂N₂O | 9.53 | Positive | 343.0399 | 307.06327 | 271.08659 | 139.98977 | 96.04439 | |
| Buprofezin | C₁₆H₂₃N₃OS | 10.84 | Positive | 306.1635 | 201.10561 | 116.05285 | 106.06513 | 145.04301 | 86.06004 |
| Butylate | C₁₁H₂₃NOS | 10.81 | Positive | 218.1573 | 156.13829 | 162.09471 | 100.07569 | 125.09609 | |
| Chlorfluazuron | C₂₀H₉Cl₃F₅N₃O₃ | 11.25 | Positive | 539.9702 | 382.93632 | 141.01465 | 186.95862 | 158.0412 | |
| Cumyluron | C₁₇H₁₉ClN₂O | 9.79 | Positive | 303.1259 | 185.04762 | 119.08553 | 125.01525 | 91.05423 | 73.03964 |
| Cyanazine | C₉H₁₃ClN₆ | 7.93 | Positive | 241.0963 | 214.0854 | 96.05562 | 104.001 | 132.0323 | 174.0541 |
| Cycluron | C₁₁H₂₂N₂O | 9.03 | Positive | 199.1805 | 72.04439 | 89.07094 | 69.06988 | 111.11683 | |
| Cyproconazole | C₁₅H₁₈ClN₃O | 10.09 | Positive | 292.1211 | 70.03997 | 125.01525 | | | |
| Cyromazine | C₆H₁₀N₆ | 2.54 | Positive | 167.104 | 85.05087 | 125.08217 | 108.05562 | 83.06037 | 60.05562 |
| Diclobutrazol | C₁₅H₁₉C₁₂N₃O | 10.2 | Positive | 328.0978 | 70.03997 | 158.97628 | | | |
| Diflufenican | C₁₉H₁₁F₅N₂O₂ | 10.69 | Positive | 395.0813 | 266.04234 | 238.04742 | 169.05222 | 145.02596 | 96.04439 |
| Dimepiperate | C₁₅H₂₁NOS | 10.66 | Positive | 264.1417 | 146.06341 | 119.08553 | 91.05423 | 112.07569 | 69.06988 |
| Dimethametryn | C₁₁H₂₁N₅S | 9.89 | Positive | 256.159 | 186.08079 | 96.05562 | 71.06037 | 116.02769 | 68.02432 |
| Dimethenamid | C₁₂H₁₈ClNO₂S | 9.47 | Positive | 276.082 | 168.08415 | 244.05574 | 126.0372 | 111.0263 | 152.05285 |
| Dimethomorph | C₂₁H₂₂ClNO₄ | 9.58 | Positive | 388.131 | 301.0626 | 165.05462 | 138.99452 | 273.06768 | 70.02874 |
| Diniconazole | C₁₅H₁₇Cl₂N₃O | 10.58 | Positive | 326.0821 | 70.03997 | 158.97628 | 172.95555 | | |
| Diphenamid | C₁₆H₁₇NO | 9.09 | Positive | 240.1383 | 134.09643 | 167.08553 | 165.06988 | 152.06205 | 91.05423 |
| Dipropetryn | C₁₁H₂₁N₅S | 9.89 | Positive | 256.159 | 214.11209 | 144.03384 | 172.06514 | 186.08079 | 228.12774 |
| Diuron | C₉H₁₀Cl₂N₂O | 9.11 | Positive | 233.0243 | 72.04439 | 159.97153 | | | |
| Dodemorph | C₁₈H₃₅NO | 9.1 | Positive | 282.2791 | 116.10699 | 98.09643 | 97.10118 | 83.08553 | |
| Epoxiconazole | C₁₇H₁₃ClFN₃O | 9.94 | Positive | 330.0804 | 121.0448 | 123.02407 | 91.05423 | 70.03997 | 141.01017 |
| Ethiprole | C₁₃H₉C₁₂F₃N₄OS | 9.46 | Positive | 396.9899 | 254.96981 | 350.94803 | 212.94802 | 227.95892 | |
| Etobenzanid | C₁₆H₁₅CLNO₃ | 10.39 | Positive | 340.0502 | 179.07027 | 149.05986 | 121.02841 | 282.00831 | |
| Fenbuconazole | C₁₉H₁₇ClN₄ | 10.01 | Positive | 337.1215 | 70.03997 | 125.01525 | 91.05423 | | |
| Fenpropathrin | C₂₂H₂₃NO₃ | 11.2 | Positive | 350.1751 | 125.09609 | 181.06479 | 97.10118 | | |
| Fenpropimorph | C₂₀H₃₃NO | 9.32 | Positive | 304.2635 | 147.11683 | 117.06988 | 132.09335 | 119.08553 | 105.06988 |
| Fenpyroximate | C₂₄H₂₇N₃O₄ | 11.23 | Positive | 422.2074 | 138.06619 | 366.14483 | 231.10023 | 135.04406 | 214.09749 |
| Fenuron | C₉H₁₂N₂O | 6.52 | Positive | 165.1022 | 72.04439 | 120.04439 | 92.04948 | | |
| Flufenoxuron | C₂₁H₁₁ClF₆N₂O₃ | 11.13 | Positive | 489.0435 | 158.0412 | 141.01465 | 306.03033 | | |
| Fluometuron | C₁₀H₁₁F₃N₂O | 8.69 | Positive | 233.0896 | 72.04439 | 160.03686 | 168.02555 | | |
| Flurtamone | C₁₈H₁₄F₃NO₂ | 9.45 | Positive | 334.1049 | 247.07291 | 105.03349 | 178.07774 | 227.06665 | |
| Flusilazole | C₁₆H₁₅F₂N₃Si | 10.08 | Positive | 316.1076 | 187.05819 | 165.06969 | | | |
| Flutolanil | C₁₇H₁₆F₃NO₂ | 9.62 | Positive | 324.1206 | 242.0611 | 262.06741 | 173.02088 | 282.07364 | |
| Flutriafol | C₁₆H₁₃F₂N₃O | 8.84 | Positive | 302.1099 | 70.03997 | 123.02407 | 233.07725 | 109.0448 | |
| Forchlorfenuron | C₁₂H₁₀ClN₃O | 9.07 | Positive | 248.0585 | 129.0214 | 155.00067 | 137.03473 | | |
| Imazalil | C₁₄H₁₄Cl₂N₂O | 8.74 | Positive | 297.0556 | 158.97628 | 255.00864 | 200.98685 | 172.99193 | 81.04472 |
| Imibenconazole | C₁₇H₁₃Cl₃N₄S | 10.93 | Positive | 410.9999 | 125.01525 | 341.96723 | 171.00307 | 295.97951 | 251.01527 |
| Isoproturon | C₁₂H₁₈N₂O | 8.94 | Positive | 207.1492 | 72.04439 | 165.10224 | 134.09643 | | 125.00268 |
| Linuron | C₉H₁₀Cl₂N₂O₂ | 9.53 | Positive | 249.0192 | 159.97153 | 182.02414 | 160.97936 | 132.96063 | 125.00268 |
| Metconazole | C₁₇H₂₂ClN₃O | 10.43 | Positive | 320.1524 | 70.03997 | 125.01525 | | | |
| Methabenzthiazuron | C₁₀H₁₁N₃OS | 8.97 | Positive | 222.0696 | 165.0481 | 150.02462 | 124.02155 | 109.01065 | 92.04948 |
| Methoprotryne | C₁₁H₂₁N₅OS | 8.88 | Positive | 272.154 | 198.08079 | 240.12774 | 230.10701 | 212.09644 | 170.04949 |
| Metobromuron | C₉H₁₁BrN₂O₂ | 8.88 | Positive | 259.0077 | 169.95999 | 148.06311 | 170.96781 | 91.04165 | 119.06037 |
| Metoxuron | C₁₀H₁₃ClN₂O₂ | 7.63 | Positive | 229.0738 | 72.04439 | 156.02107 | | | |
| Monolinuron | C₉H₁₁ClN₂O₂ | 8.64 | Positive | 215.0582 | 126.0105 | 148.06311 | 127.01833 | 98.9996 | |
| Myclobutanil | C₁₅H₁₇ClN₄ | 9.72 | Positive | 289.1215 | 70.03997 | 125.01525 | 151.0309 | 220.08875 | 164.02615 |
| Neburon | C₁₂H₁₆Cl₂N₂O | 10.22 | Positive | 275.0712 | 88.11208 | 114.09134 | 57.06988 | 58.02874 | 187.96645 |
| Paclobutrazol | C₁₅H₂₀ClN₃O | 9.6 | Positive | 294.1368 | 70.03997 | 125.01525 | 87.08044 | | |
| Penconazole | C₁₃H₁₅Cl₂N₃ | 10.25 | Positive | 284.0716 | 70.03997 | 158.97628 | 172.99193 | | |
| Phenmedipham | C₁₆H₁₆N₂O₄ | 9.2 | Positive | 301.1183 | 136.0393 | 168.06552 | 108.04439 | 111.04406 | |
| Picoxystrobin | C₁₈H₁₆F₃NO₄ | 10.09 | Positive | 368.1104 | 145.06479 | 115.05423 | 130.04132 | 91.05423 | 117.06988 |
| Prochloraz | C₁₅H₁₆Cl₃N₃O₂ | 10.36 | Positive | 376.0381 | 308.00064 | 265.95369 | 222.94787 | 70.02874 | 70.06513 |
| Prometon | C₁₀H₁₉N₅O | 8.41 | Positive | 226.1662 | 142.07234 | 184.11929 | 100.05054 | 68.02432 | |
| Prometryn | C₁₀H₁₉N₅S | 9.43 | Positive | 242.1434 | 158.04949 | 200.09644 | 116.02769 | 68.02432 | 110.04612 |
| Propazine | C₉H₁₆ClN₅ | 9.38 | Positive | 230.1167 | 146.0228 | 188.06975 | 104.001 | 110.04612 | |
| Propiconazole | C₁₅H₁₇Cl₂N₃O₂ | 10.3 | Positive | 342.0771 | 158.97628 | 204.98176 | 186.9712 | | |
| Pymetrozine | C₁₀H₁₁N₅O | 3.72 | Positive | 218.1036 | 105.04472 | 79.04127 | 78.03344 | | |
| Pyraclostrobin | C₁₉H₁₈ClN₃O₄ | 10.41 | Positive | 388.1059 | 163.06278 | 164.0706 | 194.08117 | 296.05852 | 149.04713 |
| Pyriproxyfen | C₂₀H₁₉NO₃ | 11.03 | Positive | 322.1438 | 96.04439 | 227.10666 | 185.05971 | 134.07262 | 136.07569 |
| Sebuthylazine | C₉H₁₆ClN₅ | 9.51 | Positive | 230.1167 | 174.0541 | 96.05562 | 132.0323 | 104.001 | 68.02432 |
| Secbumeton | C₁₀H₁₉N₅O | 8.41 | Positive | 226.1662 | 170.10364 | 100.05054 | 142.07234 | 128.08184 | 96.05562 |
| Simeconazole | C₁₄H₂₀FN₃OSi | 9.92 | Positive | 294.1432 | 70.03997 | 135.06045 | 73.0468 | 209.07925 | |
| Simetryn | C₈H₁₅N₅S | 8.14 | Positive | 214.1121 | 124.0871 | 96.05562 | 144.05899 | 116.02769 | 166.10872 |
| Tebuconazole | C₁₆H₂₂ClN₃O | 10.26 | Positive | 308.1524 | 70.03397 | 125.01525 | 151.0309 | | |
| Tebuthiuron | C₉H₁₆N₄OS | 8.24 | Positive | 229.1118 | 116.02769 | 172.09029 | 157.06682 | 74.0059 | 62.0059 |
| Terbuthylazine | C₉H₁₆ClN₅ | 9.38 | Positive | 230.1167 | 174.0541 | 96.05562 | 132.0323 | 146.0228 | 104.001 |
| Terbumeton | C₁₀H₁₉N₅O | 8.41 | Positive | 226.1662 | 170.10364 | 142.07234 | 100.05054 | | |
| Terbutryn | C₁₀H₁₉N₅S | 9.53 | Positive | 242.1434 | 186.08079 | 68.02432 | 71.06037 | 96.05562 | 116.02769 |
| Tetraconazole | C₁₃H₁₁C₁₂F₄N₃O | 9.92 | Positive | 372.0288 | 158.97628 | 70.03997 | 184.99193 | | |
| Thenylchlor | C₁₆H₁₈ClNO₂S | 9.9 | Positive | 324.082 | 127.02121 | 97.01065 | 65.03858 | 58.995 | |
| Triadimefon | C₁₄H₁₆ClN₃O₂ | 9.68 | Positive | 294.1004 | 197.07277 | 141.01017 | 126.99452 | 129.01017 | |
| Triadimenol | C₁₄H₁₈ClN₃O₂ | 9.79 | Positive | 296.116 | 70.03997 | 99.08044 | 227.08333 | | |
| Tridemorph | C₁₉H₃₉NO | 10.27 | Positive | 298.3104 | 98.09643 | 130.12264 | 116.10699 | 71.08553 | 57.06988 |
| Trifloxystrobin | C₂₀H₁₉F₃N₂O₄ | 10.59 | Positive | 409.137 | 186.05251 | 145.02596 | 116.04948 | 206.08117 | 146.06004 |
| Triticonazole | C₁₇H₂₀ClN₃O | 9.87 | Positive | 318.1368 | 70.03997 | 191.0622 | 125.01525 | | |
| Acetochlor | C₁₄H₂₀ClNO₂ | 10 | Positive | 270.1255 | 238.09932 | 162.12773 | 148.11208 | 224.08367 | 59.04914 |
| Alachlor | C₁₄H₂₀ClNO₂ | 11.01 | Positive | 270.1255 | 238.09932 | 162.12773 | 90.0105 | 220.08875 | 76.97887 |
| Allidochlor | C₈H₁₂ClNO | 7.66 | Positive | 174.068 | 98.09643 | 106.00542 | 81.06988 | 79.05423 | 105.06988 |
| Butachlor | C₁₇H₂₆ClNO₂ | 10.71 | Positive | 312.1725 | 252.11497 | 176.14338 | 147.10425 | 118.06513 | |
| Butralin | C₁₄H₂₁N₃O₄ | 11.29 | Positive | 296.1605 | 240.09788 | 222.08732 | 208.07167 | | |
| Chlordimeform | C₁₀H₁₃ClN₂ | 6.11 | Positive | 197.084 | 117.0573 | 125.01525 | 152.02615 | 89.03858 | |
| Dazomet | C₅H₁₀N₂S₂ | 4.49 | Positive | 163.0358 | 119.99362 | 90.0372 | 95.04919 | 135.04414 | 133.02849 |
| Dinitramine | C₁₁H₁₃F₃N₄O₄ | 10.54 | Positive | 323.0962 | 305.0856 | 247.04374 | 215.06648 | 261.05939 | |
| Diphenylamine | C₁₂H₁₁N | 9.99 | Positive | 170.0964 | 93.05785 | | | | |
| Dithiopyr | C₁₅H₁₆F₅NO₂S₂ | 10.73 | Positive | 402.0615 | 354.05817 | 271.99922 | 296.03554 | 312.01122 | 360.01459 |
| Fenoxanil | C₁₅H₁₈Cl₂N₂O₂ | 10.1 | Positive | 329.0818 | 302.07091 | 86.09643 | 188.98685 | 141.11482 | |
| Florasulam | C₁₂H₈F₃N₅O₃S | 7.49 | Positive | 360.0373 | 129.03846 | 191.99265 | 109.03278 | | |
| Flumetsulam | C₁₂H₉F₂N₅O₂S | 6.66 | Positive | 326.0518 | 129.0384572 | 144.02555 | 191.992532 | | |
| Haloxyfop-methyl | C₁₆H₁₃ClF₃NO₄ | 10.56 | Positive | 376.0558 | 316.03467 | 272.00845 | 91.05423 | 288.08936 | 119.04914 |
| Hexazinone | C₁₂H₂₀N₄O₂ | 8.17 | Positive | 253.1659 | 171.08765 | 71.06037 | 85.07602 | | |
| Isopropalin | C₁₅H₂₃N₃O₄ | 11.38 | Positive | 310.1761 | 226.08223 | 268.12918 | 250.11862 | | |
| Isoprothiolane | C₁₂H₁₈O₄S₂ | 9.61 | Positive | 291.0719 | 188.96746 | 144.97763 | 231.01441 | 172.97255 | 84.97426 |
| Isoxaben | C₁₈H₂₄N₂O₄ | 9.55 | Positive | 333.809 | 165.05462 | 122.03636 | 150.03115 | 107.01276 | |
| Metazachlor | C₁₄H₁₆ClN₃O | 8.87 | Positive | 278.1055 | 134.09643 | 210.06802 | 105.06988 | 79.05423 | |
| Metolachlor | C₁₅H₂₂ClNO₂ | 10 | Positive | 284.412 | 252.11497 | 176.14338 | 134.09643 | 73.06479 | |
| Metosulam | C₁₄H₁₃Cl₂N₅O₄S | 8.33 | Positive | 418.0138 | 174.99501 | 140.02615 | 173.98718 | 189.9820958 | |
| Metamitron | C₁₀H₁₀N₄O | 6.6 | Positive | 203.0927 | 104.04948 | 175.09782 | 79.05423 | | |
| Norflurazon | C₁₂H₉ClF₃N₃O | 9.01 | Positive | 304.0459 | 284.03967 | 160.03686 | 87.99483 | 264.03344 | 140.03063 |
| Ofurace | C₁₄H₁₆ClNO₃ | 8.2 | Positive | 282.0891 | 254.09423 | 236.08367 | 160.11208 | 224.08367 | 178.12264 |
| Picolinafen | C₁₉H₁₂F₄N₂O₂ | 10.99 | Positive | 377.0908 | 238.04742 | 256.05807 | 359.0802 | 145.02596 | |
| Pretilachlor | C₁₇H₂₆ClNO₂ | 10.71 | Positive | 312.1725 | 252.11497 | 176.14338 | | | |
| Propachlor | C₁₁H₁₄ClNO | 8.93 | Positive | 212.0837 | 170.03672 | 94.06513 | 106.06513 | 152.02615 | |
| Propanil | C₉H₉Cl₂NO | 9.58 | Positive | 218.0134 | 161.98718 | 127.01833 | | | |
| Propisochlor | C₁₅H₂₂ClNO₂ | 10 | Positive | 284.1412 | 212.08367 | 176.14338 | 252.11497 | 224.12005 | 73.06479 |
| Pyrazoxyfen | C₂₀H₁₆Cl₂N₂O₃ | 10.26 | Positive | 403.0611 | 91.05423 | 105.03349 | 119.04914 | 157.06091 | 285.01921 |
| Pyrifenox | C₁₄H₁₂Cl₂N₂O | 9.7 | Positive | 295.0399 | 93.0573 | 263.01373 | 67.04165 | | |
| Pyroquilon | C₁₁H₁₁NO | 8.13 | Positive | 174.0913 | 132.08078 | 117.0573 | 105.06988 | 120.08078 | 130.06513 |
| Tepraloxydim | C₁₇H₂₄ClNO₄ | 9.8 | Positive | 342.1467 | 250.14377 | 166.08626 | 98.06004 | 222.14886 | |
| Thiazopyr | C₁₆H₁₇F₅N₂O₂S | 10.22 | Positive | 397.1004 | 377.09414 | 335.04719 | 61.01065 | 303.02093 | 317.09065 |
| Tralkoxydim | C₂₀H₂₇NO₃ | 11.07 | Positive | 330.2064 | 138.05495 | 284.16451 | 140.0706 | 164.0706 | 267.16177 |
| Allethrin | C₁₉H₂₆O₃ | 10.93 | Positive | 303.1955 | 123.11683 | 135.08044 | 107.08553 | 93.06988 | 169.12231 |
| Anilofos | C₁₃H₁₉ClNO₃PS₂ | 10.28 | Positive | 368.0305 | 170.96978 | 198.9647 | 124.98206 | | |
| Aspon | C₁₂H₂₈O₅P₂S₂ | 11.23 | Positive | 379.0926 | 210.90481 | 114.96156 | 252.95176 | 294.99871 | 138.99771 |
| Bromfenvinphos-ethyl | C₁₂H₁₄BrCl₂O₄P | 10.37 | Positive | 402.9263 | 98.98417 | 169.96846 | 155.04695 | 127.01565 | 248.88679 |
| Cadusafos | C₁₀H₂₃O₂PS₂ | 10.57 | Positive | 271.095 | 158.96978 | 130.93848 | 215.03238 | | |
| Chlorpyrifos | C₉H₁₁Cl₃NO₃PS | 11.12 | Positive | 349.9336 | 197.92747 | 114.96142 | 293.87096 | 321.90226 | 171.02393 |
| Coumaphos | C₁₄H₁₆ClO₅PS | 10.39 | Positive | 363.0217 | 226.99263 | 306.95913 | 334.99043 | 288.94857 | 211.01565 |
| Crufomate | C₁₂H₁₉ClNO₃P | 10.06 | Positive | 292.0864 | 126.03146 | 236.02378 | 260.06017 | 57.06988 | |
| Cycloate | C₁₁H₂₁NOS | 10.64 | Positive | 216.1417 | 83.08553 | 154.12264 | 134.06341 | 72.04439 | 63.0263 |
| Cyphenothrin | C₂₄H₂₅NO₃ | 11.3 | Positive | 376.1907 | 151.11174 | 123.11683 | 181.06479 | 208.07569 | 81.06988 |
| DEF(Tribufos) | C₁₂H₂₇OPS₃ | 11.4 | Positive | 315.1034 | 168.99052 | 112.92792 | 259.04084 | 225.05312 | |
| Demeton S-methyl | C₆H₁₅O₃PS₂ | 8.26 | Positive | 231.0273 | 89.04195 | 61.01065 | 101.02332 | 114.96133 | 142.99263 |
| Demeton S-sulfone | C₆H₁₅O₅PS₂ | 7.15 | Positive | 263.0171 | 98.98417 | 234.98583 | 140.97698 | 95.01613 | 127.01547 |
| Dichlorvos | C₄H₇Cl₂O₄P | 8.13 | Positive | 220.9532 | 127.0155 | 144.98173 | 112.99982 | 78.99409 | |
| Ditalimfos | C₁₂H₁₄NO₄PS | 9.95 | Positive | 300.0454 | 148.0393 | 243.98279 | 272.01409 | 130.02888 | 114.96144 |
| EPTC(eptam) | C₉H₁₉NOS | 10.19 | Positive | 190.126 | 128.10699 | 86.06004 | 162.13118 | | |
| Ethion | C₉H₂₂O₄P₂S₄ | 11 | Positive | 384.9949 | 142.93848 | 170.96978 | 114.96151 | 199.00108 | |
| Etrimfos | C₁₀H₁₇N₂O₄PS | 10.3 | Positive | 293.0719 | 265.04064 | 142.99263 | 233.01443 | | |
| Famphur | C₁₀H₁₆NO₅PS₂ | 8.84 | Positive | 326.028 | 217.00832 | 280.97018 | 142.99263 | 93.00999 | |
| Fenamiphossulfone | C₁₃H₂₂NO₅PS | 8.34 | Positive | 336.1029 | 118.04732 | 266.02466 | 108.05706 | 308.07161 | 139.02121 |
| Fenamiphossulfoxide | C₁₃H₂₂NO₄PS | 8.21 | Positive | 320.108 | 171.0474268 | 250.0297416 | 292.07669 | 154.04469 | |
| Fensulfothion | C₁₁H₁₇O₄PS₂ | 8.93 | Positive | 309.0379 | 114.96133 | 171.02393 | 142.99263 | 247.02221 | |
| Fenthion | C₁₀H₁₅O₃PS₂ | 9.53 | Positive | 279.0273 | 231.02405 | 149.02347 | 216.00055 | 153.03702 | |
| Fenthionsulfone | C₁₀H₁₅O₅PS₂ | 8.59 | Positive | 311.0171 | 142.99269 | 127.01549 | 78.99438 | 200.00557 | |
| Fenthionsulfoxide | C₁₀H₁₅O₄PS₂ | 8.4 | Positive | 295.0222 | 127.01564 | 279.99902 | 278.01947 | 142.99263 | 200.00554 |
| Heptenophos | C₉H₁₂ClO₄P | 9.04 | Positive | 251.0234 | 127.01547 | 125.01525 | 89.03858 | | |
| Iprobenfos | C₁₃H₂₁O₃PS | 10.13 | Positive | 289.1022 | 91.05423 | 205.00828 | 65.03858 | | |
| Isazophos | C₉H₁₇ClN₃O₃PS | 9.77 | Positive | 314.049 | 162.06619 | 114.9615 | 119.06069 | 178.04334 | |
| Isocarbamid | C₈H₁₅N₃O₂ | 7.12 | Positive | 186.1237 | 87.05529 | 130.0611 | | | |
| Malathion | C₁₀H₁₉O₆PS₂ | 9.65 | Positive | 331.0433 | 99.00764 | 127.03898 | 142.99266 | | |
| Molinate | C₉H₁₇NOS | 9.76 | Positive | 188.1104 | 126.09134 | 83.08553 | 98.09643 | | |
| Pebulate | C₁₀H₂₁NOS | 10.57 | Positive | 204.1417 | 162.09471 | 128.10699 | 72.04439 | 57.06988 | 106.03211 |
| Phenthoate | C₁₂H₁₇O₄PS₂ | 10.19 | Positive | 321.0379 | 135.04406 | 107.04914 | 163.07536 | 91.05423 | 79.05423 |
| Phorate | C₇H₁₇O₂PS₃ | 10.54 | Positive | 261.0201 | 75.0263 | 114.9614 | 142.93848 | | |
| Phosalone | C₁₂H₁₅ClNO₄PS₂ | 10.51 | Positive | 367.9941 | 182.00033 | 139.00595 | 138.0105 | 114.94149 | 130.04018 |
| Phosphamidon | C₁₀H₁₉ClNO₅P | 7.7 | Positive | 300.0762 | 127.01547 | 174.06802 | 100.07569 | 226.98706 | |
| Phoxim | C₁₂H₁₅N₂O₃PS | 10.47 | Positive | 299.0614 | 129.04472 | 95.04914 | 105.04472 | 215.98807 | 114.96127 |
| Piperophos | C₁₄H₂₈NO₃PS₂ | 10.6 | Positive | 354.1321 | 255.0273 | 170.9334 | 142.93848 | 212.98035 | |
| Pirimiphos-methyl | C₁₁H₂₀N₃O₃PS | 10.43 | Positive | 306.1036 | 108.05562 | 164.11822 | 142.99263 | 136.08692 | 67.02907 |
| Profenophos | C₁₁H₁₅BrClO₃PS | 10.8 | Positive | 372.9424 | 302.86417 | 114.9615 | 344.91112 | 143.9795 | 128.00234 |
| Propetamphos | C₁₀H₂₀NO₄PS | 9.75 | Positive | 282.0923 | 138.0137 | 156.02426 | 194.98754 | 222.03483 | |
| Pyraclofos | C₁₄H₁₈ClN₂O₃PS | 10.44 | Positive | 361.0537 | 256.98773 | 138.01042 | 140.02646 | | |
| Pyrazophos | C₁₄H₂₀N₃O₅PS | 10.47 | Positive | 374.0934 | 228.08737 | 238.0645 | 346.0621 | 194.05602 | |
| Pyridaphenthion | C₁₄H₁₇N₂O₄PS | 9.72 | Positive | 341.0719 | 189.06585 | 313.04064 | 92.04948 | 285.00934 | 114.96127 |
| Quinalphos | C₁₂H₁₅N₂O₃PS | 10.28 | Positive | 299.0614 | 147.0552 | 163.03245 | 114.96133 | 242.99878 | 271.03008 |
| Resmethrin | C₂₂H₂₆O₃ | 11.48 | Positive | 339.1955 | 171.08044 | 143.08553 | 91.05423 | 128.06206 | 293.18999 |
| Tebupirimphos | C₁₃H₂₃N₂O₃PS | 10.93 | Positive | 319.124 | 153.10224 | 277.07703 | 231.03516 | 249.04573 | |
| Terbufos | C₉H₂₁O₂PS₃ | 10.92 | Positive | 289.0514 | 57.06988 | 103.0576 | 187.00108 | 158.96978 | 130.93848 |
| Terbufossulfone | C₉H₂₁O₄PS₃ | 9.35 | Positive | 321.0412 | 114.96133 | 171.02393 | 142.99263 | 128.97698 | |
| Tetrachlorvinphos | C₁₀H₉C₁₄O₄P | 10.11 | Positive | 364.9065 | 127.01556 | 238.89834 | 203.92948 | | |
| Tolclofos-methyl | C₉H₁₁Cl₂O₃PS | 10.59 | Positive | 300.9616 | 142.99263 | 286.94598 | 174.97119 | 127.01548 | 78.99434 |
| Triazophos | C₁₂H₁₆N₃O₃PS | 9.79 | Positive | 314.0723 | 162.06619 | 119.06037 | 114.96133 | | |
| Trietazine | C₉H₁₆ClN₅ | 9.91 | Positive | 230.1167 | 99.09167 | 132.0323 | 202.0854 | 104.001 | 71.06037 |
| Vernolate | C₁₀H₂₁NOS | 10.57 | Positive | 204.1417 | 128.10699 | 86.06004 | | | |
| Chloridazon | C₁₀H₈ClN₃O | 6.8 | Positive | 222.0429 | 104.04948 | 92.04948 | 65.03858 | 128.98502 | 146.01157 |
| Flumiclorac-pentyl | C₂₁H₂₃ClFNO₅ | 10.83 | Positive | 424.1322 | 308.04843 | 326.05899 | 107.04914 | 135.04454 | 3540539 |
| Imazamethabenz-methyl | C₁₆H₂₀N₂O₃ | 8.11 | Positive | 289.1547 | 144.04439 | 86.09643 | 161.07094 | 229.13354 | 257.12845 |
| Acibenzolar S-methyl | C₈H₆N₂OS₂ | 9.7 | Positive | 210.9994 | 136.00897 | 167.96981 | | | |
| Butocarboxim | C₇H₁₄N₂O₂S | 7.27 | Positive | 191.0849 | 75.0263 | 116.05285 | | | |
| Piperonylbutoxide | C₁₉H₃₀O₅ | 11.02 | Positive | 339.2166 | 177.09101 | 119.08553 | 149.05971 | 147.08044 | |
| Propham | C₁₀H₁₃NO₂ | 9.12 | Positive | 180.1019 | 120.04439 | 138.05495 | 92.05006 | | |
| Fludioxonil | C₁₂H₆F₂N₂O₂ | 9.4 | Positive | 249.047 | 180.03282 | 126.03477 | 169.0406 | 151.03003 | |
| Hexaflumuron | C₁₆H₈Cl₂F₆N₂O₃ | 10.7 | Positive | 460.9889 | 158.0412 | 141.01465 | | | |
| Novaluron | C₁₇H₉ClF₈N₂O₄ | 10.7 | Positive | 493.0196 | 158.0412 | 141.01456 | 310.0064 | | |
| Cyclanilide | C₁₁H₉Cl₂NO₃ | 9.87 | Positive | 274.0032 | 113.02381 | | | | |
| Nitralin | C₁₃H₁₉N₃O₆S | 9.95 | Positive | 346.1067 | 141.11482 | | | | |
| Terbacil | C₉H₁₃ClN₂O₂ | 8.39 | Positive | 217.0738 | 161.01123 | 143.98468 | 163.0082816 | 145.9817325 | |
| Chlorthiophos | C₁₁H₁₅Cl₂O₃PS₂ | 11.64 | Positive | 360.965 | 304.9024 | 332.9337 | 224.93607 | 191.95615 | |
| Cypermethrin | C₂₂H₁₉Cl₂NO₃ | 11.94 | Positive | 416.0815 | 191.0025 | 127.0309 | 91.05423 | 163.00758 | 181.06479 |
| Demeton-S | C₈H₁₉O₃PS₂ | 9 | Positive | 259.0586 | 89.04195 | 61.01065 | | | |
| (Disulfotonoxon) | | | | | | | | | |
| Phoratesulfoxide | C₇H₁₇O₄PS₂ | 6.57 | Positive | 261.0379 | 128.97698 | 171.02393 | 243.0273 | | |
| Thiometon | C₆H₁₅O₂PS₃ | 8.43 | Positive | 247.0045 | 61.01065 | 89.04195 | | | |
| Imazaquin | C₁₇H₁₇N₃O₃ | 7.55 | Positive | 312.1343 | 199.0502 | 86.09643 | 267.1128 | 252.07675 | 69.06988 |
| Chlorpyrifos-methyl | C₇H₇Cl₃NO₃PS | 10.7 | Positive | 321.9023 | 142.99263 | 289.87605 | 78.99441 | 127.01556 | |
| Benzoylprop-ethyl | C₂₀H₂₂ClNO₅ | 10.19 | Positive | 392.1259 | 105.03349 | 95.04915 | 77.03858 | | |
| Thifensulfuron methyl | C₁₂H₁₃N₅O₆S₂ | 8.17 | Positive | 388.038001 | 167.05635 | 141.07709 | 222.97294 | | |
| Chlorotoluron | C₁₀H₁₃ClN₂O | 8.88 | Positive | 213.0789173 | 72.04439 | 140.02615 | 168.02107 | 125.01525 | 94.0652 |
| Trimethacarb | C₁₁H₁₅NO₂ | 9.04 | Positive | 194.1175553 | 137.09609 | 122.07262 | | | |
| Clomazone | C₁₂H₁₄ClNO₂ | 9.33 | Positive | 240.078583 | 125.01525 | 127.01233 | | | |
| Bromuconazole(cis-) | C₁₃H₁₂BrCl₂N₃O | 9.85 | Positive | 375.9613566 | 158.97628 | 172.95555 | | | |
| Mecarbam | C₁₀H₂₀NO₅PS₂ | 9.98 | Positive | 330.0593271 | 142.93848 | 226.996 | 199.00108 | 170.96978 | |
| Propaquizafop | C₂₂H₂₂ClN₃O₅ | 10.95 | Positive | 444.132075 | 100.07569 | 299.05818 | 371.07919 | 255.03197 | 70.06527 |
| Aldicarb-NH4 | C₇H₁₄N₂O₂S | 7.5 | Positive | 208.11142 | 89.04195 | 116.05285 | 70.06513 | | |
| Aldoxycarb(Aldicarb | C₇H₁₄N₂O₄S | 4.67 | Positive | 240.10125 | 86.06004 | 148.04268 | 76.03941 | 166.05324 | 223.0747 |
| Sulfone)-NH4 | | | | | | | | | |
| Atraton | C₉H₁₇N₅O | 7.71 | Positive | 212.15059 | 170.10364 | 100.05054 | 114.06619 | 142.07234 | 128.08184 |
| Atrazine-Desethyl | C₆H₁₀ClN₅ | 7 | Positive | 188.06975 | 146.0228 | 104.001 | 110.04612 | | |
| Azinphos-Ethyl | C₁₂H₁₆N₃O₃PS₂ | 9.8 | Positive | 346.04435 | 137.00556 | 132.04439 | 232.94867 | 95.04945 | 114.96133 |
| Aziprotryne | C₇H₁₁N₇S | 9.59 | Positive | 226.08694 | 156.03384 | 125.08217 | 114.01204 | 89.0168 | 198.08079 |
| Benzofenap | C₂₂H₂₀Cl₂N₂O₃ | 10.58 | Positive | 431.09237 | 105.06988 | 157.06077 | 119.04914 | 133.06479 | |
| Bioallethrin | C₁₉H₂₆O₃ | 10.75 | Positive | 303.19547 | 135.08044 | 169.12231 | 151.11174 | 123.11683 | 107.08553 |
| Bioresmethrin | C₂₂H₂₆O₃ | 11.31 | Positive | 339.19547 | 171.08044 | 143.08553 | 128.06205 | 105.03349 | 225.12739 |
| Bolstar | C₁₂H₁₉O₂PS₃ | 10.88 | Positive | 323.03576 | 139.02121 | 154.9715 | 218.96978 | 200.95922 | 171.97424 |
| Bromacil | C₉H₁₃BrN₂O₂ | 8.11 | Positive | 261.02332 | 204.96072 | 187.93417 | 161.9549 | 143.94434 | 98.04746 |
| Brompyrazon | C₁₀H₈BrN₃O | 6.91 | Positive | 265.99235 | 92.04948 | 104.04948 | 131.94434 | 172.9345 | |
| Chlorimuron Ethyl | C₁₅H₁₅ClN₄O₆S | 9.5 | Positive | 415.04736 | 186.00599 | 213.02092 | 121.02841 | 83.02448 | |
| Chlormequat Chloride | C₅H₁₂ClN | 0.8 | Positive | 122.0731 | 86.09643 | 94.0418 | 106.0418 | | |
| Chlorphoxim | C₁₂H₁₄ClN₂O₃PS | 10.27 | Positive | 333.0224 | 114.96133 | 163.00527 | 111.04406 | 139.00558 | 86.9996 |
| Chlorsulfuron | C₁₂H₁₂ClN₅O₄S | 8.38 | Positive | 358.03713 | 141.07709 | 167.05635 | 100.05054 | 129.01017 | |
| Cinidon-Ethyl | C₁₉H₁₇Cl₂NO₄ | 10.66 | Positive | 394.06074 | 348.01888 | 366.02944 | 107.04914 | 135.04406 | 239.96087 |
| Cinosulfuron | C₁₅H₁₉N₅O₇S | 7.85 | Positive | 414.1078 | 183.05127 | 157.072 | 121.02841 | 215.03657 | |
| Coumatetralyl | C₁₉H₁₆O₃ | 9.79 | Positive | 293.11722 | 175.03897 | 131.08553 | 107.04914 | 163.03897 | 215.07027 |
| Crimidine | C₇H₁₀ClN₃ | 6.98 | Positive | 172.0636 | 136.08692 | 109.07602 | 95.06037 | 119.06037 | |
| Cyclosulfamuron | C₁₇H₁₉N₅O₆S | 9.75 | Positive | 422.11288 | 261.02813 | 139.03763 | 279.03869 | 236.03288 | 199.08257 |
| Cythioate | C₈H₁₂NO₅PS₂ | 7.66 | Positive | 297.99673 | 142.99263 | 217.00828 | 283.98108 | 124.02155 | 108.04439 |
| Deet(Diethyltoluamide) | C₁₂H₁₇NO | 8.82 | Positive | 192.13829 | 119.04914 | 109.06479 | 100.07569 | 164.10699 | |
| Desmetryne | C₈H₁₅N₅S | 8.16 | Positive | 214.11209 | 172.06514 | 82.03997 | 124.06117 | 116.02769 | 99.06652 |
| Dialifos | C₁₄H₁₇ClNO₄PS₂ | 10.4 | Positive | 394.00979 | 208.01598 | 181.00504 | 158.96978 | 130.93848 | 114.96133 |
| Diallate(Total) | C₁₀H₁₇Cl₂NOS | 10.55 | Positive | 270.04807 | 108.96063 | 128.10699 | 86.06004 | 228.00112 | 142.94835 |
| Dichlofenthion | C₁₀H₁₃C₁₂O₃PS | 9.28 | Positive | 314.97728 | 258.91468 | 286.94598 | 114.96136 | 222.93801 | 162.9712 |
| Dichlofluanid | C₉H₁₁Ch₂FN₂O₂S₂ | 9.8 | Positive | 332.96958 | 223.94983 | 123.01379 | 188.98053 | 153.0043 | |
| Dichlorobenzamide | C₆H₃CONH₂Cl₂ | 5.2 | Positive | 189.9821 | 172.95555 | 108.98395 | 146.97628 | 120.96063 | 128.00234 |
| Dicyclohexyl Phthalate | C₂₀H₂₆O₄ | 11.1 | Positive | 331.19039 | 167.03389 | 149.02332 | 249.11214 | 121.02841 | |
| Dimefox | C₄H₁₂FN₂OP | 6.06 | Positive | 155.0744 | 110.01656 | 135.06818 | 92.02598 | | |
| Dimethirimol | C₁₁H₁₉N₃O | 7.19 | Positive | 210.16009 | 140.10699 | 98.06004 | 150.10257 | 165.10224 | 122.09643 |
| Dimethyl Phthalate | C₁₀H₁₀O₄ | 7.9 | Positive | 195.06519 | 163.03897 | 135.04406 | 105.03349 | | |
| Dioxabenzofos | C₈H₉O₃PS | 8.93 | Positive | 217.00828 | 107.04914 | 123.0263 | 137.04195 | 154.9715 | 186.99771 |
| Disulfoton Sulfone | C₈H₁₉O₄PS₃ | 8.78 | Positive | 307.02558 | 114.96133 | 171.02393 | 260.98372 | 142.99263 | 232.95242 |
| Dmst | C₉H₁₄N₂O₂S | 8.32 | Positive | 215.08487 | 106.06513 | 136.0995 | 151.12298 | | |
| Dodine | C₁₃H₂₉N₃ | 10.43 | Positive | 228.24342 | 186.22163 | 85.10118 | 57.07061 | 71.0861 | |
| Emamectin B1A Benzoate | C₄₉H₇₅NO₁₃ | 10.77 | Positive | 886.53112 | 158.11756 | 82.06513 | 126.09134 | 302.1962 | |
| Emamectin B1B Benzoate | C₄₈H₇₃NO₁₃ | 10.6 | Positive | 872.51547 | 158.11756 | 82.06513 | 126.09134 | 302.1962 | |
| Etaconazole | C₁₄H₁₅Cl₂N₃O₂ | 9.8 | Positive | 328.06141 | 158.97628 | 204.98176 | 186.9712 | 245.01306 | 259.02871 |
| Ethiofencarb Sulfoxide | C₁₁H₁₅NO₃S | 6.33 | Positive | 242.08454 | 107.0491414 | 185.0630769 | 164.0706 | 95.04914 | |
| Ethoprophos (Ethoprop) | C₈H₁₉O₂PS₂ | 9.84 | Positive | 243.06368 | 172.98543 | 130.93848 | 215.03238 | 201.01673 | 138.99771 |
| Ethoxysulfuron | C₁₅H₁₈N₄O₇S | 9.56 | Positive | 399.0969 | 279.03938 | 236.03288 | 182.05602 | 139.03763 | 109.02841 |
| Ethylene Thiourea | C₃H₆N₂S | 1.62 | Positive | 103.03245 | 86.0059 | 57.07052 | | | |
| Fenfuram | C₁₂H₁₁NO₂ | 8.51 | Positive | 202.08626 | 109.02841 | 120.04439 | 184.07569 | 92.04948 | |
| Fentrazamide | C₁₆H₂₀ClN₅O₂ | 10.15 | Positive | 350.13783 | 197.12845 | 154.12264 | 115.0502 | | |
| Flamprop-Isopropyl | C₁₉H₁₉ClFNO₃ | 10.19 | Positive | 364.11103 | 105.03349 | 172.03189 | 276.05811 | 304.05302 | |
| Flamprop-Methyl | C₁₇H₁₅ClFNO₃ | 9.62 | Positive | 336.07973 | 105.03349 | 95.04963 | 276.0586 | 304.05351 | |
| Fluazifop-Butyl | C₁₉H₂₀F₃NO₄ | 10.62 | Positive | 384.14172 | 282.0725 | 328.07798 | 238.04628 | 164.03177 | 119.04914 |
| Fluazuron | C₂₀H₁₀C₁₂F₅N₃O₃ | 10.82 | Positive | 506.00921 | 158.0412 | 348.97415 | 141.01445 | 123.99485 | |
| Flubenzimine | C₁₇H₁₀F₆N₄S | 10.61 | Positive | 417.06031 | 187.04776 | 167.04153 | 136.02135 | 95.04963 | |
| Fluorochloridon | C₁₂H₁₀Cl₂F₃NO | 9.6 | Positive | 312.01643 | 292.0102 | 212.06816 | 240.06307 | 172.03686 | |
| Fluridone | C₁₉H₁₄F₃NO | 9.12 | Positive | 330.11003 | 310.1038 | 290.09757 | 242.09643 | 115.05423 | |
| Fluthiacet-Methyl | C₁₅H₁₅ClFN₃O₃S₂ | 10.13 | Positive | 404.03002 | 344.00889 | 273.99721 | 214.98393 | 309.03849 | |
| Fonofos | C₁₀H₁₅OPS₂ | 9.31 | Positive | 247.03747 | 137.01845 | 108.98715 | 200.9956 | 80.95585 | 126.9977 |
| Furmecyclox | C₁₄H₂₁NO₃ | 10.13 | Positive | 252.15942 | 170.08117 | 110.06004 | 138.05496 | 97.06479 | |
| Guthion(Azinphos-Methyl) | C₁₀H₁₂N₃O₃PS₂ | 9.1 | Positive | 318.01305 | 142.99278 | 132.04439 | 260.9806 | 104.04948 | |
| Haloxyfop-2-Etotyl | C₁₉H₁₉ClF₃NO₅ | 10.57 | Positive | 434.09766 | 316.03418 | 272.00797 | 298.02362 | 388.0558 | |
| Imazapic | C₁₄H₁₇N₃O₃ | 7.05 | Positive | 276.13427 | 163.0502 | 216.07675 | 231.1128 | 188.08184 | |
| Imazethapyr | C₁₅H₁₉N₃O₃ | 7.73 | Positive | 290.14992 | 177.06585 | 245.12845 | 230.0924 | 159.05529 | |
| Isofenphos | C₁₅H₂₄NO₄PS | 10.29 | Positive | 346.12364 | 121.02841 | 216.97189 | 245.00319 | 137.00587 | |
| Isomethiozin | C₁₂H₂₀N₄OS | 10.26 | Positive | 269.14306 | 200.08521 | 172.09029 | 116.02769 | 130.04334 | |
| Isouron | C₁₀H₁₇N₃O₂ | 8.24 | Positive | 212.13935 | 167.0815 | 128.08184 | 140.04545 | 111.0189 | |
| Kadethrin-NH4 | C₂₃H₂₄O₄S | 10.54 | Positive | 414.17336 | 171.08044 | 143.08553 | 128.06205 | 283.07873 | |
| Kresoxim-Methyl | C₁₈H₁₉NO₄ | 10.08 | Positive | 314.13868 | 222.09134 | 235.07536 | 116.04948 | 194.09643 | |
| Malaoxon | C₁₀H₁₉O₇PS | 8.16 | Positive | 315.06619 | 99.00767 | 127.03897 | 142.99263 | 113.02332 | |
| Mefenpyr-Diethyl | C₁₆H₁₈Cl₂N₂O₄ | 10.2 | Positive | 373.07164 | 327.0297742 | 159.9715311 | 271.00356 | 132.96063 | |
| Mephosfolan | C₈H₁₆NO₃PS₂ | 7.96 | Positive | 270.0382 | 167.98788 | 139.95658 | 75.0263 | 242.0069 | |
| Metalaxyl-M | C₁₅H₂₁NO₄ | 8.77 | Positive | 280.15433 | 192.13829 | 160.11208 | 220.13321 | 248.12812 | |
| Methfuroxam | C₁₄H₁₅NO₂ | 9.08 | Positive | 230.11756 | 137.05971 | 111.08044 | 120.04439 | | |
| MethylParaoxon | C₈H₁₀NO₆P | 7.73 | Positive | 248.03185 | 202.03895 | 127.01556 | 90.04646 | 234.02881 | |
| Monalide/dimethachlor | C₁₃H₁₈ClNO | 9.98 | Positive | 240.11497 | 224.08367 | 148.11208 | 132.08078 | 105.06988 | |
| Monuron | C₉H₁₁ClN₂O | 8.02 | Positive | 199.06327 | 72.04439 | 126.010502 | 154.005417 | 126.010502 | |
| Naptalam | C₁₈H₁₃NO₃ | 8.18 | Positive | 292.09682 | 149.02332 | 144.08078 | 121.02841 | 256.07559 | |
| Nicotine | C₁₀H₁₄N₂ | 2.18 | Positive | 163.12298 | 132.08078 | 130.06513 | 117.0573008 | 106.06513 | |
| Oxycarboxin | C₁₂H₁₃NO₄S | 7.04 | Positive | 268.06381 | 175.00596 | 164.98543 | 193.01674 | 104.96409 | |
| Oxydemeton Methyl | C₆H₁₅O₄PS₂ | 5.33 | Positive | 247.02221 | 169.00828 | 127.0154717 | 142.99263 | 105.03686 | |
| Phorate-Sulfone | C₇H₁₇O₄PS₃ | 8.76 | Positive | 293.00993 | 171.02397 | 142.93848 | 114.96133 | | |
| Phosfolan | C₇H₁₄NO₃PS₂ | 7.45 | Positive | 256.02255 | 139.95627 | 167.98757 | 196.01918 | 227.99125 | |
| Pirimiphos-Ethyl | C₁₃H₂₄N₃O₃PS | 10.73 | Positive | 334.13488 | 198.10594 | 182.12879 | 170.07481 | 114.96146 | |
| Pronamide | C₁₂H₁₁Cl₂NO | 9.6 | Positive | 256.02905 | 189.9820958 | 172.9555467 | 146.97628 | | |
| Propylene Thiourea | C₄H₈N₂S | 0.8 | Positive | 117.0481 | 100.02155 | 59.99025 | 71.99025 | | |
| Pyrazosulfuron-Ethyl | C₁₄H₁₈N₆O₇S | 9.76 | Positive | 415.10304 | 182.05602 | 139.0502 | 157.06077 | 213.9917 | |
| Pyrethrini 1 | C₂₁H₂₈O₃ | 11.08 | Positive | 329.21112 | 161.09609 | 143.08553 | 105.06988 | 128.06205 | |
| Pyributicarb | C₁₈H₂₂N₂O₂S | 10.84 | Positive | 331.14748 | 181.04301 | 126.05496 | 190.12264 | 151.11174 | |
| Pyriftalid | C₁₅H₁₄N₂O₄S | 9.17 | Positive | 319.0747 | 139.0502 | 157.06077 | 179.01613 | 202.03211 | |
| Pyrimidifen | C₂₀H₂₈ClN₃O₂ | 10.77 | Positive | 378.19428 | 184.06311 | 150.10257 | 169.03964 | 130.02923 | |
| Pyrithiobac-Sodium | C₁₃H₁₁ClN₂O₄S | 8.94 | Positive | 327.02008 | 309.00952 | 139.0502 | 203.9842 | 267.98297 | |
| Quinoclamine | C₁₀H₆ClNO₂ | 8.03 | Positive | 208.01598 | 172.0393 | 105.03349 | 137.01525 | 180.02107 | |
| Quizalofop-Ethyl | C₁₉H₁₇ClN₂O₄ | 10.69 | Positive | 373.09496 | 299.05769 | 271.06278 | 255.03148 | 181.01583 | |
| Rabenzazole | C₁₂H₁₂N₄ | 8.86 | Positive | 213.11347 | 172.08692 | 186.10257 | 132.0682 | 145.07602 | |
| Simeton | C₈H₁₅N₅O | 6.91 | Positive | 198.13494 | 124.08692 | 114.06619 | 100.05054 | 170.10364 | |
| Spinosad A | C₄₁H₆₅NO₁₀ | 10.21 | Positive | 732.46812 | 142.12264 | 98.09643 | 97.06479 | | |
| Spinosad D | C₄₂H₆₇NO₁₀ | 10.41 | Positive | 746.48377 | 142.12264 | 98.09643 | | | |
| Succinic Acid | C₁₂H₂₂O₄ | 10.12 | Positive | 231.15909 | 101.02332 | 115.03897 | 157.08592 | | |
| Di-N-Butyl Ester | | | | | | | | | |
| Sulfotepp | C₈H₂₀O₅P₂S₂ | 10.11 | Positive | 323.03001 | 114.96133 | 142.99263 | 171.02393 | 266.96741 | |
| Tau-Fluvalinate | C₂₆H₂₂ClF₃N₂O₃ | 11.31 | Positive | 503.13438 | 181.06479 | 208.07569 | 250.06 | | |
| Tepp | C₈H₂₀O₇P₂ | 7.82 | Positive | 291.0757 | 178.9505 | 98.98417 | 206.9818 | 235.0131 | |
| Terbucarb | C₁₇H₂₇NO₂ | 10.52 | Positive | 278.21146 | 109.06479 | 166.08626 | 222.14886 | | |
| Thiofanox-Sulfoxide | C₉H₁₈N₂O₃S | 6.24 | Positive | 235.11109 | 104.01646 | 178.08963 | 115.09917 | 160.07906 | |
| Thionazin | C₈H₁₃N₂O₃PS | 8.87 | Positive | 249.04573 | 113.0168 | 97.03964 | 192.98282 | 221.01443 | |
| Thiophanate | C₁₄H₁₈N₄O₄S₂ | 8.85 | Positive | 371.08422 | 151.03245 | 282.03654 | 134.07127 | | |
| Triallate | C₁₀H₁₆ClNOS | 11 | Positive | 304.00909 | 142.92166 | 261.96214 | 128.10699 | | |
| Triazoxide | C₁₀H₆ClN₅O | 8.87 | Positive | 248.03336 | 123.9948534 | 149.99793 | 98.9996 | 221.02246 | |
| Tributyl Phosphate | C₁₂H₂₇O₄P | 10.56 | Positive | 267.17197 | 98.98417 | 155.04677 | 211.10937 | | |
| Triisobutyl Phosphate | C₁₂H₂₇O₄P | 10.45 | Positive | 267.17197 | 98.98417 | 155.04677 | 211.10937 | | |
| Uniconazole | C₁₅H₁₈ClN₃O | 9.94 | Positive | 292.12112 | 70.04078 | 125.01525 | | | |
| Vamidothion-Sulfone | C₈H₁₈NO₆PS₂ | 5.59 | Positive | 320.03859 | 178.05324 | 169.00828 | 142.99263 | 86.06004 | |
| Vegadex(Thioallate) | C₈H₁₄ClNS₂ | 10.08 | Positive | 224.0329 | 116.05285 | 188.05622 | 150.94375 | | |

3. Qualitative Detection Method

Under the detection conditions, the accurate mass database including name of the compounds, the molecular formula, the accurate mass, the retention time and the characteristic ion fragments is established for the determined compounds of the pesticides. Then the qualitative screening method of the compounds is carried out by using TractFinder 2.5 software. The screening parameters are shown in Table 3, and the samples that meet the set range are positive samples.

TABLE 3
Screening parameter settings of TractFinder

| Use matrix Blank | 10 | |
|---|---|---|
| Mass Tolerance | 10 | ppm |
| Smoothing Point | 7 | |
| Chromatogram View Width | 2.00 | minutes |

| | |
|---|---|
| Peaks | Threshold Override: 10,000 |
| | S/N Ratio Threshold 50.0 |
| Retention time* (Identify) | Window override (see): 120 |
| Fragment Ions (Confirm) | Min. # of Fragments: Intensity |
| | Threshold: 1,000 |
| | Mass Tolerance: 10 ppm |

4. Lowest Limit for Detection

Lowest limit of the qualitative detection: the lowest limit of the qualitative detection of the method for the above 390 pesticides is 10 μg/kg.

What is claimed is:

1. A method for screening pesticide residues in medicine food homology (MFH) agricultural products comprising:
step 1, constructing a liquid chromatography-mass spectrometry database of pesticides; preparing solutions for the pesticides and using liquid chromatography and mass spectrometry to detect the solutions to obtain liquid chromatography-mass spectrograms of the solutions, wherein a detection condition of the liquid chromatography comprises:
a liquid chromatography column: an Accucore™ aQ C18 hydrophilic interaction liquid chromatography (HPLC) column, 150 millimeter (mm)×2.1 mm, a particle size of 2.6 micrometer (μm);
a mobile phase A: ammonium formate of 5 millimol per liter (mM) and a 0.1% formic acid aqueous solution;
a mobile phase B: ammonium formate of 5 Mm and a 0.1% formic acid methanol solution;
a column temperature: 25° C.;
wherein a detection condition of the mass spectrometry comprises:
an ion source: an electrospray ion source;
a temperature of the ion source: 325° C.;
a temperature of a heater: 350° C.;
a sheath gas flow rate: 40 atmospheres relative to vacuum (arb);
an aux gas flow rate: 10 arb;
step 2, processing a sample to obtain a processed sample, and detecting the processed sample to obtain a liquid chromatography-mass spectrogram of the processed sample;
step 3, comparing and analyzing the liquid chromatography-mass spectrogram obtained from step 2 with the liquid chromatography-mass spectrograms in the liquid chromatography-mass spectrometry database obtained from step 1 individually.

2. The method for screening the pesticide residues in the MFH agricultural products as claimed in claim 1, wherein the liquid chromatography-mass spectrometry database comprises: names of compounds, molecular formulas of the compounds, masses of the compounds, retention times of the compounds and masses of characteristic ion fragments.

3. The method for screening the pesticide residues in the MFH agricultural products as claimed in claim 2, wherein the pesticides in step 1 comprise at least one selected from the group consisting of acephate, acetamiprid, aminocarb, azamethiphos, benalaxyl, bendiocarb, benfuracarb, bensulide, benzoximate, bifenazate, bupirimate, butafenacil, butoxycarboxim, buturon, carpropamid, carbaryl, carbendazim, carbetamide, carbofuran, carboxin, clethodim, clothianidin, cyazofamid, cycloxydim, cyprodinil, desmedipham, dicrotophos, diethofencarb, dimethoate, dinotefuran, dioxycarb, ethiofencarb, ethirimol, ethofumesate, fenazaquin, fenhexamid, fenobucarb, fenoxycarb, fenpropidine, flufenacet, fuberidazole, furalaxyl, furathiocarb, hexythiazox, hydramethylnon, imidachloprid, indoxacarb, isoprocarb, mefenacet, mepanipyrim, mepronil, metalaxyl, methamidophos, methomyl, methoxyfenozide, m-tolyl methylcarbamate, metribuzin, napropamide, nitenpyram, nuarimol, omethoate, oxamyl, pencycuron, primicarb, propamocarb, propargite, propoxur, prosulfocarb, pyrimethanil, quinoxyfen, rotenone, spirodiclofen, tebufenozide, tebutam, temephos, thiabendazole, thiacloprid, thiamethoxam, thiobencarb, thiodicarb, thiophanate-methyl, trichlorfon, vamidothion, ametryn, atrazine, azoxystrobin, benodanil, benoxacor, bitertanol, boscalid, buprofezin, butylate, chlorfluazuron, cumyluron, cyanazine, cycluron, cyproconazole, cyromazine, diclobutrazole, diflufenican, dimepiperate, dimethametryn, dimethenamid, dimethomorph, diniconazole, diphenamid, dipropetryn, diuron, dodemorph, epoxiconazole, ethiprole, etobenzanid, fenbuconazole, fenpropathrin, fenpropimorph, fenpyroximate, fenuron, flufenoxuron, fluometuron, flurtamone, flusilazole, flutolanil, flutriafol, forchlorfenuron, imazalil, imibenconazole, isoproturon, linuron, metconazole, methabenzthiazuron, methoprotryne, metobromuron, metoxuron, monolinuron, myclobutanil, neburon, paclobutrazole, penconazole, phenmedipham, picoxystrobin, prochloraz, prometon, prometryne, propazine, propiconazole, pymetrozine, pyraclostrobin, pyriproxyfen, sebutylazine, secbumeton, simeconazole, simetryn, tebuconazole, tebuthiuron, terbuthylazine, terbumeton, terbutryne, tetraconazole, thenylchlor, triadimefon, triadimenol, tridemorph, trifloxystrobin, triticonazole, acetochlor, alachlor, allidochlor, butachlor, butralin, chlordimeform, dazomet, dinitramine, diphenylamine, dithiopyr, fenoxanil, florasulam, flumetsulam, haloxyfop-2-ethoxyethyl, hexazinone, isopropalin, isoprothiolane, isoxaben, metazachlor, metolachlor, metosulam, metamitron, norflurazon, ofurace, picolinafen, pretilachlor, propachlor, propanil, propisochlor, prosulfocarb, pyrifenox, pyroquinolone, tepraloxydim, thiazopyr, tralkoxydim, allethrin, anilofos, aspon, bromfenvinfos, cadusafos, chlorpyrifos, coumaphos, crufomate, cycloate, cyphenothrin, defoliant, demeton-S-methyl, demeton-S-methylsulphone, dichlorvos, ditalimfos, eradicane, ethion, etrimfos, famphur, fenamiphossulfone, fenamiphos metabolite, fensulfothion, fenthion, fenthionsulfone, fenthionsulfoxide, heptenephos, iprobenfos, isazofos, isocarbamid, malathion, molinate, pebulate, phenthoate, phorate, phosalone, phosphamidon, phoxim, piperophos, pirimiphos-methyl, profenofos, propetamphos, pyraclofos, pyrazophos, pyridaphenthion, quinalphos, resmethrin, tebupirimfos, terbufos, terbufos sulfone, tetrachlorvinphos, tolclofos-methyl, triazophos, trietazine, vernolate, chloridazon, flumiclorac-pentyl, imazamethabenz-methyl, benzothiadiazole, butocarboxim, piperonyl-butoxide, propham, fludioxonil, hexaflumuron, fluorourea, cyclanilide, nitralin, terbacil, chlorthiophos, cypermethrin, demeton, phorate sulfoxide, thiometon, imazaquin, chlorpyrifos-methyl, benzoylprop, thifensulfuron-methyl, chlortoluron, trimethacarb, clomazone, bromuconazole (cis), mecarbam, propaquizafop, aldicarb, aldicarb sulfoxide, atratone, deethylatrazine, azinphos-ethyl, aziprotryn, benzofenap, bioallethrin, bioresmethrin, sulprofos, bromacil, brompyrazon, chlorimuron, cycocel, chlorpoxime, chlorsulfuron, cinidon-ethyl, cinosulfuron, coumatetralyl, cyclosulfamuron, cythioate, diethyltoluamide, desmetryne, dialifos, diallate, dichlofenthion, dichlofluanid, dichlorobenzamide, dicyclohexyl phthalate, dimefox, dimethirimol, dimethyl phthalate, dioxabenzofos, disulfoton-sulfone, dimethylbenzenesulfonamide, dodine, emamectin benzoate-Bla, emamectin benzoate-B1b, etaconazole, ethiofencarb-sulfoxide, ethoprophos, ethoxysulfuron, ethylene thiourea, ofurace, fentrazamide, flamprop-isopropyl, flamprop-methyl, fluazifop-butyl, fluazuron, flubenzimine, flurochloridone, fluridone, fluthiacet, fonofos, furmecyclox, azinphos-methyl, pyrflumetocloprid, imazapic, imazethapyr, isofenphos, isomethiozin, isouron, kadethrin, kresoxim-methyl, malaoxon, mefenpyr-diethyl, mephosfolan, mefenoxam, methfuroxam, paraoxon-methyl, monalide, monuron, naptalam, nicotine, oxycarboxin, oxydemeton-methyl, phorate-sulfone, phosfolan, pirimiphos-ethyl, propyzamide, propylenethiourea, pyrazosulfuron methyl, pyrethrin 1, pyributicarb, pyriftalid, pyrimidifen, pyrithiobac-sodium, quinoclamine, quizalofop, rabenzazole, simeton, pleocidin A, pleocidin D, tabatrex, sulfotepp, tau-fluvalinate, tetraethyl diphosphate, terbucarb, thiofanox sulfoxide, thionazin, thiophanate, triallate, triazoxide, tri-n-butyl phosphate, tri-iso-butyl phosphate, uniconazole, vamidothion sulfone and sulfallate.

4. The method for screening the pesticide residues in the MFH agricultural products as claimed in claim 3, wherein a flow rate of the liquid chromatography is a range of 0.35-0.45 milliliters per minute (mL/min);
wherein a condition of gradient elution is as follows: an initial proportion of the mobile phase A is 100%, the initial proportion of the mobile phase A gradually decreases to a first proportion of 80% within 4 minutes, the first proportion gradually decreases to a second proportion of 60% within 1.5 minutes, the second proportion gradually decreases to a third proportion of 0% within 4 minutes, following by remaining for 2.4 minutes, and then the third proportion gradually increases to the initial proportion of 100%.

5. The method for screening the pesticide residues in the MFH agricultural products as claimed in claim 4, wherein the flow rate of the liquid chromatography is 0.4 mL/min.

6. The method for screening the pesticide residues in the MFH agricultural products as claimed in claim 5, wherein a scanning range of the mass spectrometry comprises: 100-900 mass-to-charge ratios (M/Z) in a positive ion mode, or 100-700 M/Z in a negative ion mode.

7. The method for screening the pesticide residues in the MFH agricultural products as claimed in claim 6, wherein a resolution of the mass spectrometry comprises: 70000 in full mass spectrometry scanning, or 17500 in tandem mass spectrometry (MS/MS).
